(19) United States
(12) Patent Application Publication
(10) Pub. No.: US 2005/0014774 A1
(43) Pub. Date: Jan. 20, 2005

(54) Oxo-pyrimidine compounds

(76) Inventors: Richard Storer, Kent (GB); Adel Moussa, Burlington, MA (US); Paolo La Colla, Cagliari (IT); Marino Artico, Roma (IT)

Correspondence Address: KING & SPALDING LLP, 191 PEACHTREE STREET, N.E., ATLANTA, GA, 30303-1763, US

(21) Appl. No.: 10/833,601

(22) Filed: Apr. 28, 2004

Related U.S. Application Data
(60) Provisional application No. 60/466,195, filed on Apr. 28, 2003.

(51) Int. Cl. A61K 31/513

(52) U.S. Cl. 514/269; 544/319; 544/320

(57) ABSTRACT

Oxo-pyrimidine compounds for the treatment of retroviral infections, and particularly for HIV, are described. Also included are compositions comprising the oxo-pyrimidine derivatives alone or in combination with other anti-retroviral agents, processes for their preparation, and methods of manufacturing a medicament incorporating these compounds.

### CROSS-REFERENCE OF THE INVENTION

This application claims priority to U.S. Provisional Application No. 60/466,195, filed Apr. 28, 2003, which is incorporated by reference.

### FIELD OF THE INVENTION

The present invention is in the field of optionally substituted oxo-pyrimidine compounds. It also provides processes for making the compounds and methods of using these compounds for the treatment of subjects infected with Human Immunodeficiency Virus (HIV). It further provides pharmaceutical compositions that include compounds as active agents, and to the use of these compounds in the manufacture of medicaments for treating subjects who suffer from HIV infection.

### BACKGROUND OF THE INVENTION

Human immunodeficiency virus, or HIV, is the retroviral agent responsible for the complex disease that includes progressive degeneration of the central and peripheral nervous system, and destruction of the immune system known as Acquired Immune Deficiency Syndrome or AIDS. Since it emerged as a public health threat in 1983, numerous efforts have focused on options for treating, controlling and/or eradicating the disease.

A focal point in AIDS research efforts has been the development of inhibitors of human immunodeficiency virus (HIV-1) reverse transcriptase (RT), the enzyme responsible for the reverse transcription of retroviral RNA to proviral DNA (W. C. Greene, New Engl. J. Med., 1991, 324:308-17; Mitsuya et al., Science, 1990, 249:1533-44; E. DeClerq, J. Acquired Immune Defic. Syndr. Res. Human Retrovirus, 1992, 8:119-134). The HIV virus infects human T-4 cells that coordinate the immune system, and destroys them or effectively changes their normal function. Thus, an infected subject suffers from a continually decreasing number of T-4 cells, and the T-4 cells that remain exhibit abnormal activity. As a result, the immune system is unable to combat infections and cancers, and the HIV-infected subject usually succumbs to them. HIV infection is associated with other conditions including Kaposi's sarcoma, thrombocytopenia, AIDS-related complex (ARC), peripheral neuropathy, lymphadenopathy, and central nervous system infection that produces disorientation, ataxia, and progressive disarthria.

Promising inhibitors of HIV include non-nucleoside reverse transcriptase inhibitors (NNRTIs). NNRTIs bind to a specific allosteric site of the HIV-1 transcriptase near the polymerase site and interfere with reverse transcription by altering either the conformation or mobility of the reverse transcriptase. In turn, this leads to non-competitive inhibition of the enzyme (Kohlstaedt et al., Science, 1992, 256:1783-90).

Several classes of compounds have been identified as being NNRTIs. Examples include the following:
1) 1-[(2-hydroxyethoxy)methyl]-6-(phenylthio)thymines or HEPTS (Tanaka et al., J. Med. Chem., 1991, 34:349-57; Pontikis et al., J. Med. Chem., 1997, 40:1845-54; Danel et al., J. Med. Chem., 1996, 39:2427-31; Baba et al., Antiviral Res., 1992, 17:245-64);
2) bis(heteroaryl)piperazines or BHAPs (Romero et al., J. Med. Chem., 1993, 36:1505-08);
3) dihydroalkoxybenzyloxopyrimidines or DABOs (Danel et al., Acta Chemica Scandinavica, 1997, 51:426-30; Mai et al., J. Med. Chem., 1997, 40:1447-54; Sudbeck et al., Antimicrobial Agents Chemotherapy, 1998, 42(12):3225-33; LaColla and Artico, U.S. Pat. No. 6,635,636 and WO 00/03998; Sommadossi, LaColla, Artico and Bryant, U.S. Pat. No. 6,545,007 and WO 02/40021);
4) 2′, 5′-bis-O-(tertbutyldimethylsilyl)-3′-spiro-5″-(4″-amino-1″, 2″-oxathiole-2″, 2″-dioxide) pyrimidines or TSAOs (Balzarini et al., Proc. Natl. Acad. Sci. USA, 1992, 89:4392-96);
5) phenethylthiazolylthiourea (PETT) derivatives (Bell et al., J. Med. Chem., 1995, 38:4929-36; Cantrell et al., J. Med. Chem., 1996, 39:4261-74);
6) tetrahydro-imidazo[4,5,1-jk][1,4]-benzodiazepine-2(1H)-one and -thione (TIBO) derivatives (Pauwels et al., Nature, 1990, 343:470-4);
7) alpha-anilinophenylacetamide (alpha-APA) derivatives (Pauwels et al., Proc. Natl. Acad. Sci. USA, 1993, 90:1711-15);
8) arylthio and bithiobisarylamide derivatives (Domagala et al., U.S. Pat. No. 5,929,114); and
9) indole derivatives (Williams et al., U.S. Pat. No. 5,527,819; Williams et al., WO 94/19321). In addition, see U.S. Pat. No. 5,124,327, issued Jun. 23, 1992 to Greenlee et al. and assigned to Merck disclosed a class of compounds of the general formula above, in which X is H, R⁶ is H, Y is S, and R is phenyl. The patent disclosed that the compounds act as reverse transcriptase inhibitors.

U.S. Pat. No. 6,710,068 to Idenix Pharmaceuticals, Ltd., disclosed a class of phenylindoles substituted with at least two moieties other than hydrogen on either the phenyl ring or the benzyl ring of the indole function, or on both rings. The substituents are preferably contained at the 3″ and 5″ positions if located on phenyl ring, and at the 4′ and 5′; 5′ and 6′ or the 5′ and 7′ positions if located on the benzyl ring of the indole function. See also WO 02/083126.

Variously substituted dihydroalkoxy-benzyloxopyrimidines (DABOs) have been found to be effective against HIV when included as the active agent in pharmaceutical preparations administered by different routes, including oral (LaColla and Artico, U.S. Pat. No. 6,635,636 and WO 00/03998) and topical (Sommadossi, LaColla, Artico and Bryant, U.S. Pat. No. 6,545,007 and WO 02/40021) applications.

In 1999, Janssen Pharmaceutica N.V. (“Janssen”) disclosed pyrimidine derivatives that were effective in inhibiting HIV replication in patients infected with the virus (WO 99/50250; EP 0 945 433 A1). These compounds were variously substituted pyrimidines bound to an optionally substituted phenyl or pyridine moiety via a nitrogen-linking group. Janssen reported structurally similar pyrimidine derivative compounds that utilized optionally substituted pyrimidine, pyrazine or pyridazine moieties in addition to the previously reported aryl and pyridine groups bound via a nitrogen-linking group to the core pyrimidine (WO 00/27825; WO 01/85699; WO 01/22938 A1). Janssen disclosed the use of optionally substituted triazines as a core compound moiety (WO 01/85700 A2). These compounds also were found to be effective against HIV replication. In the Janssen publications, the predominant compounds were variously substituted diaryl-pyrimidine derivatives.

Ludovici et al. disclosed the synthesis and anti-HIV-1 activity of a series of substituted diarylpyrimidine (DAPY) derivative compounds as a class of non-nucleoside reverse transcriptase inhibitors, or NNRTIs (Ludovici et al., Bioorganic & Med. Chem. Letters, 2001, 11:2235-9). Several members of this class of compounds exhibited potency against wild-type as well as single mutant and double mutant strains of HIV-1.

Other compounds that bear structural similarities to the NNRTIs reported by Janssen include 4,6-diarylpyrimidine derivatives that exhibit anti-microbial activity (Wasfy et al., Sulfur Letters, 1995, 19(2):45753), and 2,6-diarylpyrimidine cyano, sulfamyl and/or sulfonyl derivatives that exhibit anti-cancer activity (Pease et al., WO 01/64654).

To date, efforts to develop a vaccine for the prevention of HIV have not been successful. Seigel et al. reported that vaccination with inactivated SIV did not protect African Green monkeys against infection with the homologous virus, notwithstanding a strong immune response to SIV (Seigel et al., J. AIDS, 1995, 8:217-226).

It is known that over a period of time, antiviral agents that are active against HIV often induce mutations in the virus that reduce the efficacy of the drug. Drug resistance most typically occurs by mutation of a gene that encodes for an enzyme used in viral replication, and most typically in the case of HIV, the enzymes affected are reverse transcriptase, protease, or DNA integrase.

It has been demonstrated that the efficacy of an anti-HIV drug can be prolonged, augmented, or restored by administering the compound in combination or alternation with a second, and perhaps a third, antiviral compound that induces a different mutation from that caused by the principal drug. Alternatively, the pharmacokinetics, biodistribution, or other parameters of a drug can be altered by such combination or alternation therapy. In general, combination therapy is typically preferred over alternation therapy since combination therapy induces multiple simultaneous pressures on the virus. However, which mutations will be induced in the HIV-1 genome by a given drug, whether mutations are transient or permanent, or how an infected cell with a mutated HIV-1 sequence will respond to therapy in combination or alternation with other agents, cannot be predicted. These factors are exacerbated by the paucity of data relating to the kinetics of drug resistance in long-term cell cultures treated with known antiretroviral agents.

Therefore, there is a need to improve the duration of antiviral efficacy produced by antiretroviral drugs, and to provide antiviral drugs that are effective against strains of the virus that have developed cross resistance through mutational adaptation. Moreover, although many of the non-nucleoside reverse transcriptase inhibitors (NNRTIs) known in the prior art exhibit favorable pharmacokinetic and biodistribution profiles, there remains a need to provide additional drugs for therapy.

### SUMMARY OF THE INVENTION

New compounds, compositions, and methods are provided for the treatment of subjects, and in particular human subjects, infected with FIW, that exhibit significant activity against drug-resistant forms of the virus. New compounds and compositions in the manufacture of medicaments useful for treating subjects who suffer from HIV infection are also provided.

Novel classes of oxo-pyrimidine compounds have been discovered that display significant antiviral activity against HIV, and, in particular, against strains of HIV that have developed cross-resistance to other anti-HIV drugs. Surprisingly, it has been discovered that anti-HIV activity can be enhanced, or in some instances cross-resistance substantially can be overcome, by treating subjects in need thereof with compounds of the present invention. Because the compounds of the present invention exhibit anti-retroviral properties, they are useful in the treatment of mammals infected with HIV or other viruses that depend upon the enzyme, reverse transcriptase, for their replication.

In one embodiment of the present invention, the compound is represented by the chemical Formula (I): 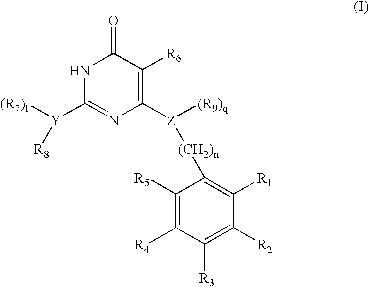
or a pharmaceutically acceptable salt, prodrug, N-oxide, quaternary amine, stereochemical isomer or tautomer thereof, wherein
a) Y is O, S, or SO₂, and t is 0; or
   Y is CH or N, and t is 1; or
   Y is C or N⁺ (with a suitable counter ion), and t is 2;
b) Z is O, S, or SO₂, and q is 0; or
   Z is CH or N, and q is 1; or
   Z is C or N⁺ (with a suitable counter ion), and q is 2;
c) n is 0-4;
d) each R₁, R₂, R₃, R₄, R₅, independently, is —H; —C₁₋₆ alkyl; —C₂₋₆ alkenyl; —C₂₋₆ alkynyl; optionally substituted aryl; -aralkyl; -halo; -haloalkyl (and —CF₃ in particular); —NO₂; —CN; —CONH₂; —CONH-C₁₋₆ alkyl; —CON(C₁₋₆ alkyl)₂; —NH₂; —NH—C₁₋₆ alkyl; —N(C₁₋₆ alkyl)₂; —NHC(O)-alkyl; —NHSO₂—C₁₋₆ alkyl; —SO₂NH₂; —SO₂NH—C₁₋₆ alkyl; —SO₂N(C₁₋₆ alkyl)₂; —SO₂NH-aryl; and —OW or —SW, where W is —H, or optionally substituted alkyl, aryl or aralkyl;
e) R₆ is —H; -halo; —C₁₋₆ straight or branched chain alkyl; —C₁₋₆ alkenyl; —C₁₋₆ alkynyl; —C₃₋₆ cycloalkyl; 3-7 membered heterocycle; -aryl; -aralkyl; -halo; -haloalkyl; —CN; —CF₃; NO₂; —CONH₂; —CONH—C₁₋₆ alkyl; —CON(C₁₋₆ alkyl)₂; —NH₂; —NH—C₁₋₆ alkyl; —N(C₁₋₆ alkyl)₂; —NHC(O)—C₁₋₆ alkyl; —NHSO₂—C₁₋₆ alkyl; —SO₂NH₂; —SO₂NH—C₁₋₆ alkyl; —SO₂N(C₁₋₆ alkyl)₂; —SO₂NH-aryl; any of which may be substituted or unsubstituted; or
   R₆ is —OW or —SW when Z is N or preferably C, and where W is —H or optionally substituted alkyl, aryl or aralkyl;
f) each R₇ and R₈ independently, is —H; —C₁₋₆ straight or branched chain alkyl; —C₂₋₆ alkenyl; —C₂₋₆ alkynyl; —C₃₋₆ cycloalkyl; 3-7 membered heterocycle; -aralkyl; -aryl; -haloalkyl; —CN; —CONH₂; —CONH—C₁₋₆ alkyl; —CON(C₁₋₆ alkyl)₂; —NH₂; —NH—C₁₋₆ alkyl; —N(C₁₋₆ alkyl)₂; —NHC(O)—C₁₋₆ alkyl; —NHSO₂—C₁₋₆ alkyl; —(CH₂)n-phenyl; —SO₂NH₂; —SO₂NH—C₁₋₆ alkyl; —SO₂N(C₁₋₆ alkyl)₂; —SO₂NH-aryl; any of which may be substituted or unsubstituted; or
   each R₇ and R₈ independently, is —OW or —SW, when Z is N or preferably C, and where W is —H or optionally substituted alkyl, aryl or aralkyl; or
   R₇ and R₈ together with the Y substituent to which they are attached, form an alicyclic, aryl or heterocyclic ring that optionally may have one or more O, S or N atoms, and further may be substituted or unsubstituted;
g) R₉ is —H; —C₁₋₆ straight or branched chain alkyl; —C₁₋₆ alkenyl; —C₁₋₆ alkynyl; —C₃₋₆ cycloalkyl; 3-7 membered heterocycle; -aryl; -aralkyl; -haloalkyl; CN; —CONH₂; —CONH—C₁₋₆ alkyl; —CON(C₁₋₆ alkyl)₂; —NH₂; —NH—C₁₋₆ alkyl; —N(C₁₋₆ alkyl)₂; —NHC(O)—C₁₋₆ alkyl; —NHSO₂—C₁₋₆ alkyl; —SO₂NH₂; —SO₂NH—C₁₋₆ alkyl; —SO₂N(C₁₋₆ alkyl)₂; —SO₂NH-aryl; any of which may be substituted or unsubstituted; or
   R₉ is —OW or —SW when Z is N or preferably C, and where W is —H or optionally substituted alkyl, aryl or aralkyl; or
h) alternatively, R₆ and R₉ taken together with the pyrimidinone ring and Z substituent to which they are attached respectively, form a bicyclic heterocycle that optionally may have additional O, S or N atoms, and further may be substituted or unsubstituted; and
   where t is 1, 2, or 3, then each occurrence of R₇ is independently selected from the substituents listed herein for R₇;
   where q is 1, 2 or 3, then each occurrence of R₉ is independently selected from the substituents listed herein for R₉.

In one preferred embodiment of Formula (I), R₆ is bromo or methyl, Z is CH, R₉ is methyl, R₁ and R₅ are both fluoro, R₃ is H or cyano, Y is N, R₇ is methyl, R₈ is ethyl, t=1, and q=1.

In a second preferred embodiment, R₆ is bromo or methyl, Z is O, R₁ and R₅ are both fluoro, R₃ is H or cyano, Y is N, R₇ is methyl, R₈ is ethyl, t=1, and q=0.

In another preferred embodiment, R₆ is bromo or methyl, Z is CH, R₉ is methyl, R₁ and R₅ are both fluoro, R₃ is H or cyano, Y is N, R₇ is methyl, R₈ is -p-cyano-phenyl, t=1, and q=1.

In yet another preferred embodiment, R₆ is bromo or methyl, Z is O, R₁ and R₅ are both methyl, R₃ is H or cyano, Y is CH, R₇ is H, R₈ is -p-cyano-phenyl, t=1, and q=0.

In still another preferred embodiment, R₆ is bromo or methyl, Z is CH, R₉ is methyl, R₁ and R₅ are both fluoro, R₃ is cyano, Y is N, R₇ is H, R₈ is -p-cyano-phenyl, t=1, and q=1.

In another preferred embodiment, R₆ is bromo or methyl, Z is CH, R₉ is methyl, R₁ and R₅ are both methyl, R₃ is cyano, Y is N, R₇ is H, R₈ is -p-cyano-phenyl, t=1, and q=1.

In still another preferred embodiment, R₆ is bromo or methyl, Z is CH, R₉ is methyl, R₁ and R₅ are both methyl, R₃ is cyano, Y is N, R₇ is H, R₈ is -p-cyano-phenyl, t=1, and q=1.

In yet another preferred embodiment, R₆ is bromo or methyl, Z is CH, R₉ is methyl, R₁ and R₅ are both fluoro, R₃ is H or cyano, Y is N, R₇ is methyl, R₈ is -p-cyano-phenyl, t=1, and q=1.

In still another preferred embodiment, R₆ is methyl, Z is CH, R₉ is methyl, R₁ and R₅ are both fluoro, R₃ is cyano, Y is N, R₇ is methyl, R₈ is ethyl, t=1, and q=1.

In yet another preferred embodiment, R₆ is bromo, Z is CH, R₉ is methyl, R₁ and R₅ are both fluoro, R₃ is H or cyano, Y is N, R₇ is methyl, R₈ is ethyl, t=1, and q=1.

In still yet another preferred embodiment, R₆ is bromo or methyl, Z is C, R₉ is dimethyl, R₁ and R₅ are both fluoro, R₃ is H or cyano, Y is N, R₇ is methyl, R₈ is ethyl, t=1, and q=1.

In still another preferred embodiment, R₆ is bromo or methyl, Z is C, R₉ is dimethyl, R₁ and R₅ are both methyl, R₃ is H or cyano, Y is N, R₇ is methyl, R₈ is ethyl, t=1, and q=1.

In yet another preferred embodiment, R₆ is bromo or methyl, Z is O, R₁ and R₅ are both methyl, R₃ is cyano, Y is N, R₇ is methyl, R₈ is ethyl, t=1, and q=0.

In still another preferred embodiment, R₆ is bromo or methyl, Z is N, R₉ is H, R₁ and R₅ are both methyl, R₃ is cyano, Y is N, R₇ is methyl, R₈ is ethyl, t=1, and q=1.

In yet still another preferred embodiment, R₆ is bromo or methyl, Z is N, R₉ is methyl, R₁ and R₅ are both methyl, R₃ is cyano, Y is N, R₇ is methyl, R₈ is ethyl, t=1, and q=1.

In still another preferred embodiment, R₆ is bromo or methyl, Z is SO₂, R₁ and R₅ are both methyl, R₃ is cyano, Y is N, R₇ is methyl, R₈ is ethyl, t=1, and q=0.

In yet another preferred embodiment, R₆ is bromo or methyl, Z is O, R₁ and R₅ are both fluoro, R₃ is H or cyano, Y is N, R₇ is methyl, R₈ is ethyl, t=1, and q=0.

In still yet another preferred embodiment, R₆ is bromo or methyl, Z is N, R₉ is H, R₁ and R₅ are both fluoro, R₃ is H or cyano, Y is N, R₇ is methyl, R₈ is ethyl, t=1, and q=1.

In still yet another preferred embodiment, R₆ is bromo or methyl, Z is N, R₉ is methyl, R₁ and R₅ are both fluoro, R₃ is H or cyano, Y is N, R₇ is methyl, R₈ is ethyl, t=1, and q=1.

In another preferred embodiment, R6 is bromo or methyl, Z is SO₂, R₁ and R₅ are both fluoro, R₃ is H or cyano, Y is N, R₇ is methyl, R₈ is ethyl, t=1, and q=0.

In yet another preferred embodiment, R₆ is bromo or methyl, Z is C, R₉ is dimethyl, R₁ and R₅ are both fluoro, R₃ is H or cyano, Y is N, R₇ is methyl, R₈ is ethyl, t=1, and q=2.

In still other preferred embodiments, R₈ is optionally substituted —(CH₂)ᵣ-phenyl and the compounds of the present invention are represented by the Formula (Ia): 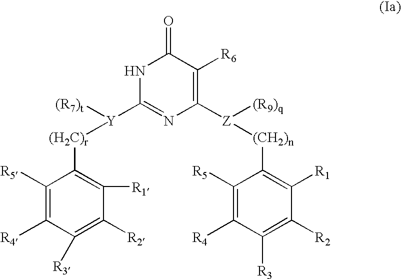
or a pharmaceutically acceptable salt, prodrug, N-oxide, quaternary amine, stereochemical isomer or tautomer thereof, wherein
a) each R₁, R₂, R₃, R₄, R₅, R_{1′}, R_{2′}, R_{3′}, R_{4′}, and R_{5′} independently, is —H; —C₁₋₆ alkyl; —C₂₋₆ alkenyl; —C₂₋₆ alkynyl; -aralkyl; optionally substituted aryl; -halo; -haloalkyl; —NO₂; —CN; —CONH₂; —CONH—C₁₋₆ alkyl; —CON(C₁₋₆ alkyl)₂; —NH₂; —NH—C₁₋₆ alkyl; —N(C₁₋₆ alkyl)₂; —NHC(O)-alkyl; —NHSO₂—C₁₋₆ alkyl; —SO₂NH₂; —SO₂NH—C₁₋₆ alkyl; —SO₂N(C₁₋₆ alkyl)₂; —SO₂NH-aryl; and —OW or —SW, where W is —H, or optionally substituted alkyl, aryl or aralkyl;
b) R₆ and R₇, each independently, is H; C₁₋₆ straight or branched chain alkyl; —C₂₋₆ alkenyl; —C₂₋₆ alkynyl; aralkyl; halo; haloalkyl; C₃₋₆ cycloalkyl; 3-7 membered heterocycle; or -aryl; —NO₂; —CONH₂; —CONH—C₁₋₆ alkyl; —CN; —CON(C₁₋₆ alkyl)₂; —NH₂; —NH—C₁₋₆ alkyl; -N(C₁₋₆ alkyl)₂; —NHC(O)—C₁₋₆ alkyl; —NHSO₂—C₁₋₆ alkyl; —SO₂NH₂; —SO₂NH—C₁₋₆ alkyl; —SO₂N(C₁₋₆ alkyl)₂; —SO₂NH-aryl; —(CH₂)ₙ-phenyl; any of which may be substituted or unsubstituted; or
   R₆ and R₇, each independently, is —OW or —SW when Z is N or preferably C, and where W is —H or optionally substituted alkyl, aryl or aralkyl;
c) R₉ is —H; —C₁₋₆ straight or branched chain alkyl; —C₁₋₆ alkenyl; —C₁₋₆ alkynyl; —C₃₋₆ cycloalkyl; 3-7 membered heterocycle; -aryl; -aralkyl; -haloalkyl; —CN; —CONH₂; —CONH—C₁₋₆alkyl; —CON(C₁₋₆ alkyl)₂; —NH₂; —NH—C₁₋₆ alkyl; —N(C₁₋₆ alkyl)₂; —NHC(O)—C₁₋₆ alkyl; —NHSO₂—C₁₋₆ alkyl; —SO₂NH₂; —SO₂NH—C₁₋₆ alkyl; —SO₂N(C₁₋₆ alkyl)₂; —SO₂NH-aryl; any of which may be substituted or unsubstituted; or
   R₉ is —OW or —SW when Z is N or preferably C, and where W is —H or optionally substituted alkyl, aryl or aralkyl; or
d) alternatively, R₆ and R₉ taken together with the pyrimidinone ring and Z substituent to which they are attached, form a bicyclic heterocycle that optionally may have additional O, S or N atoms, and which further may be substituted or unsubstituted; and
   where t is 1, 2, or 3, then each occurrence of R₇ is independently selected from the substituents listed herein for R₇;
   where q is 1, 2 or 3, then each occurrence of R₉ is independently selected from the substituents listed herein for R₉;
e) Y is O, S, or SO₂, and t is 0; or
   Y is CH or N, and t is 1; or
   Y is C or N⁺ (with a suitable counter ion), and t is 2;
f) Z is O, S, or SO₂, and q is 0; or
   Z is CH or N, and q is 1; or
   Z is C or N⁺ (with a suitable counter ion), and q is 2;
g) n is 0-4;
h) r is 0-4.

Preferred embodiments of Formula (Ia) include the following:

In one preferred embodiment, Y is N, R₇ is H, Z is CH, R₆ is bromo or methyl, R₉ is methyl, R₁ and R₅ are both F and R₁ and R₅,are both H, or R₁ and R₅ are both H and R_{1′} and R_{5′} are both F, R₃ and R₃ are both cyano, R₂, R_{2′}, R₄ and R_{4′} are all H, t=1, q=1, r=0 or 1, and n=0 or 1.

In another preferred embodiment, Y is N, R₇ is H, Z is CH, R₆ is bromo or methyl, R₉ is methyl, R₁ and R₅ are each methyl, R₃ and R_{3′} are both cyano, R_{1′}, R_{5′}, R₂, R_{2′}, R₄ and R_{4′} are all H, t=1, q=1, r=0 or 1, and n=0 or 1.

In yet another preferred embodiment, Y is N, R₇ is H, Z is CH, R₆ is bromo or methyl, R₉ is methyl, R₁ and R₅ are each methyl, R₃ is H, R_{3′} is cyano, R_{1′}, R_{5′}, R₂, R_{2′}, R₄ and R_{4′} are all H, t=1, q=1, r=0 or 1, and n=0 or 1.

In another preferred embodiment, Y is N, R₇ is H, Z is C, R₆ is bromo or methyl, R₉ is dimethyl, R₁ and R₅ are each methyl, R₃ and R_{3′} are each H or cyano, R_{1′}, R_{5′}, R₂, R_{2′}, R₄ and R_{4′} are all H, t=1, q=2, r=0 or 1, and n=0 or 1.

In yet still another preferred embodiment, Y is N, R₇ is H, Z is C, R₆ is bromo or methyl, R₉ is dimethyl, R₁ and R₅ are each fluoro, R₃ and R_{3′} are each H or cyano, R_{1′}, R_{5′}, R₂,R_{2′}, R₄ and R_{4′} are all H, t=1, q=2, r=0 or 1, and n=0 or 1.

In still another preferred embodiment, Y is CH, Z is O, R₆ is bromo or methyl, R₁ and R₅ are each methyl, R₃ and R_{3′} are each cyano, R₇ is H, R_{1′}, R_{5′}, R₂, R_{2′}, R₄ and R_{4′} are all H, t=1, q=0, r=0 or 1, and n=0 or 1.

In yet another preferred embodiment, Y is CH, Z is N, R₉ is H, R₆ is bromo or methyl, R₇ is H, R₁ and R₅ are each methyl, R₃ and R₃ are each cyano, R_{1′}, R_{5′}, R₂, R_{2′}, R₄ and R_{4′} are all H, t=1, q=1, r=0 or 1, and n=0 or 1.

In still another preferred embodiment, Y is CH, Z is N, R₉ is methyl, R₆ is bromo or methyl, R₇ is methyl, R₁ and R₅ are each methyl, R₃ and R_{3′} are each cyano, R_{1′}, R_{5′}, R₂,R_{2′}, R₄ and R_{4′} are all H, t=1, q=1, r=0 or 1, and n=0 or 1.

In a final preferred embodiment, Y is CH, Z is SO₂, R₆ is bromo or methyl, R₇ is H, R₁ and R₅ are each methyl, R₃ and R_{3′} each cyano, and R_{1′}, R_{5′}, R₂, R_{2′}, R₄ and R_{4′} are all H, t=1, q=0, r=0 or 1, and n=0 or 1. In another embodiment, there is provided a compound of structural Formula (I): 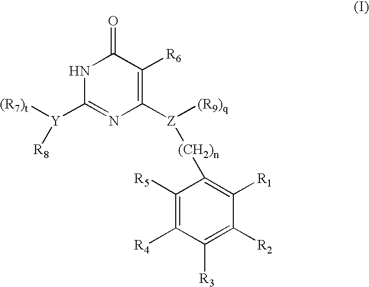
or a pharmaceutically acceptable salt, prodrug, N-oxide, quaternary amine, stereochemical isomer or tautomer thereof, wherein
a) each R₁, R₂, R₃, R₄, R₅, independently, is —H; —C₁₋₆ alkyl; —C₂₋₆ alkenyl; —C₂₋₆ alkynyl; optionally substituted aryl; -aralkyl; -halo; -haloalkyl and —CF₃ in particular; —NO₂; —CN; —CONH₂; —CONH—C₁₋₆ alkyl; —CON(C₁₋₆ alkyl)₂; —NH₂; —NH—C₁₋₆ alkyl; —N(C₁₋₆ alkyl)₂; —NHC(O)-alkyl; —NHSO₂—C₁₋₆ alkyl; —SO₂NH₂; —SO₂NH—C₁₋₆ alkyl; —SO₂N(C₁₋₆ alkyl)₂; —SO₂NH-aryl; and —OW or —SW, where W is —H, or optionally substituted alkyl, aryl or aralkyl;
b) R₇ and R₈, each independently, is —H; —C₁₋₆ straight or branched chain alkyl; —C₂₋₆ alkenyl; —C₂₋₆ alkynyl; —C₃₋₆ cycloalkyl; 3-7 membered heterocycle; -aralkyl; -aryl; -haloalkyl; —CN; —CONH₂; —CONH—C₁₋₆ alkyl; —CON(C₁₋₆ alkyl)₂; —NH₂; —NH—C₁₋₆ alkyl; —N(C₁₋₆ alkyl)₂; —NHC(O)—C₁₋₆ alkyl; —NHSO₂—C₁₋₆ alkyl; —(CH₂)ₙ-phenyl; —SO₂NH₂; —SO₂NH—C₁₋₆ alkyl; —SO₂N(C₁₋₆ alkyl)₂; —SO₂NH-aryl; any of which may be substituted or unsubstituted; or
   R₇ and R₈, each independently, is —OW or —SW when Z is N or preferably C, and where W is —H or optionally substituted alkyl, aryl or aralkyl; or
   R₇ and R₈ together with the Y substituent to which they are attached, form an alicyclic, aryl or heterocyclic ring that optionally may have one or more O, S or N atoms, and further may be substituted or unsubstituted;
c) R₆ is —H; -halo; —C₁₋₆ straight or branched chain alkyl; —C₁₋₆ alkenyl; —C₁₋₆ alkynyl; —C₃₋₆ cycloalkyl; 3-7 membered heterocycle; -aryl; -aralkyl; -halo; -haloalkyl; —CN; —CONH₂; —CONH—C₁₋₆ alkyl; —CON(C₁₋₆ alkyl)₂; —NH₂; —NH—C₁₋₆ alkyl; —N(C₁₋₆ alkyl)₂; —NHC(O)—C₁₋₆ alkyl; —NHSO₂—C₁₋₆ alkyl; —SO₂NH₂; —SO₂NH—C₁₋₆ alkyl; —SO₂N(C₁₋₆ alkyl)₂; —SO₂NH-aryl; any of which may be substituted or unsubstituted; or
   R₆ is —OW or —SW when Z is N or preferably C, and where W is —H or optionally substituted alkyl, aryl or aralkyl;
d) R₉ is —H; —C₁₋₆ straight or branched chain alkyl; —C₁₋₆ alkenyl; —C₁₋₆ alkynyl; —C₃₋₆ cycloalkyl; 3-7 membered heterocycle; -aryl; -aralkyl; -halo; -haloalkyl; —CN; —CONH₂; —CONH—C₁₋₆ alkyl; —CON(C₁₋₆ alkyl)₂; —NH₂; —NH—C₁₋₆ alkyl; —N(C₁₋₆ alkyl)₂; —NHC(O)-C₁₋₆ alkyl; —NHSO₂—C₁₋₆ alkyl; —SO₂NH₂; —SO₂NH—C₁₋₆ alkyl; —SO₂N(C₁₋₆ alkyl)₂; —SO₂NH-aryl; any of which may be substituted or unsubstituted; or
   R₉ is —OW or —SW when Z is N or preferably C, and where W is —H or optionally substituted alkyl, aryl or aralkyl; or
e) alternatively, R₆ and R₉ taken together with the pyrimidinone ring and Z substituent to which they are attached respectively, form a bicyclic heterocycle that optionally may have additional O, S or N atoms, and further may be substituted or unsubstituted; and
   where t is 1, 2, or 3, then each occurrence of R₇ is independently selected from the substituents listed herein for R₇;
   where q is 1, 2 or 3, then each occurrence of R₉ is independently selected from the substituents listed herein for R₉;
f) Y is O, S, or SO₂, and t is 0; or
   Y is CH or N, and t is 1; or
   Y is C or N⁺ (with a suitable counter ion), and t is 2;
g) Z is O, S, or SO₂, and q is 0; or
   Z is CH or N, and q is 1; or
   Z is C or N⁺ (with a suitable counter ion), and q is 2; and
h) n is 0-4.

In one embodiment, R₈ is optionally substituted —(CH₂)ᵣ-phenyl.

In another embodiment, the compound has the structural Formula (Ia): 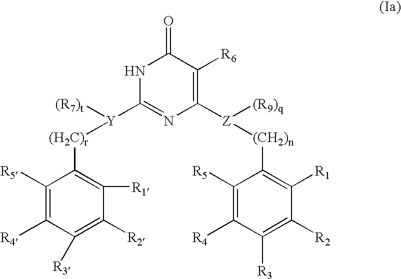
or a pharmaceutically acceptable salt, prodrug, N-oxide, quaternary amine, stereochemical isomer or tautomer thereof, wherein
a) each R₁, R₂, R₃, R₄R₅, R_{1′}, R_{2′}, R_{3′}, R_{4′}, and R_{5′} independently, is —H; —C₁₋₆ alkyl; —C₂₋₆ alkenyl; —C₂₋₆ alkynyl; -aralkyl; optionally substituted aryl; -halo; -haloalkyl and —CF₃ in particular; —NO₂; —CN; —CONH₂; —CONH—C₁₋₆ alkyl; —CON(C₁₋₆ alkyl)₂; —NH₂; —NH—C₁₋₆ alkyl; —N(C₁₋₆ alkyl)₂; —NHC(O)-alkyl; —NHSO₂—C₁₋₆ alkyl; —SO₂NH₂; —SO₂NH—C₁₋₆ alkyl; —SO₂N(C₁₋₆ alkyl)₂; —SO₂NH-aryl; and —OW or —SW, where W is —H, or optionally substituted alkyl, aryl or aralkyl;
b) R₆ and R₇, each independently, is H; C₁₋₆ straight or branched chain alkyl; —C₂₋₆ alkenyl; —C₂₋₆ alkynyl; aralkyl; halo; haloalkyl and CF₃ in particular; C₃₋₆ cycloalkyl; 3-7 membered heterocycle; or -aryl; —NO₂; —CN; —CONH₂; —CONH—C₁₋₆ alkyl; —CON(C₁₋₆ alkyl)₂; —NH₂; —NH—C₁₋₆ alkyl; —N(C₁₋₆ alkyl)₂; —NHC(O)—C₁₋₆ alkyl; —NHSO₂—C₁₋₆ alkyl; —SO₂NH₂; —SO₂NH—C₁₋₆ alkyl; —SO₂N(C₁₋₆ alkyl)₂; —SO₂NH-aryl; —(CH₂)ₙ-phenyl; any of which may be substituted or unsubstituted; or is —OW or —SW when Z is N or preferably C, and where W is —H or optionally substituted alkyl, aryl or aralkyl;
c) R₉ is —H; —C₁₋₆ straight or branched chain alkyl; —C₁₋₆ alkenyl; —C₁₋₆ alkynyl; —C₃₋₆ cycloalkyl; 3-7 membered heterocycle; -aryl; -aralkyl; -halo; -haloalkyl; —CN; —CONH₂; —CONH—C₁₋₆alkyl; —CON(C₁₋₆ alkyl)₂; —NH₂; —NH—C₁₋₆ alkyl; —N(C₁₋₆ alkyl)₂; —NHC(O)-C₁₋₆ alkyl; —NHSO₂—C₁₋₆ alkyl; —SO₂NH₂; —SO₂NH—C₁₋₆ alkyl; —SO₂N(C₁₋₆ alkyl)₂; —SO₂NH-aryl; any of which may be substituted or unsubstituted; or
   R₉ is —OW or —SW when Z is N or preferably C, and where W is —H or optionally substituted alkyl, aryl or aralkyl; or
d) alternatively, R₆ and R₉ taken together with the pyrimidinone ring and Z substituent to which they are attached, form a bicyclic heterocycle that optionally may have additional O, S or N atoms, and which further may be substituted or unsubstituted; and
   where t is 1, 2, or 3, then each occurrence of R₇ is independently selected from the substitutents listed herein for R₇;
   where q is 1, 2 or 3, then each occurrence of R₉ is independently selected from the substituents listed herein for R₉;
e) r is 0-4;
f) Y is O, S, or SO₂, and t is 0; or
   Y is CH or N, and t is 1; or
   Y is C or N⁺ (with a suitable counter ion), and t is 2;
g) Z is O, S, or SO₂, and q is 0; or
   Z is CH or N, and q is 1; or
   Z is C or N⁺ (with a suitable counter ion), and q is 2; and
h) n is 0-4.

Compositions are provided that include a compound disclosed herein in combination with a pharmaceutically acceptable carrier, diluent or excipient.

Further provided are methods of treating or preventing HIV or retroviral infection in a subject by administering an effective amount of a compound disclosed herein to a subject in need thereof.

Further provided is a method of treating or preventing HIV or retroviral infection in a subject by administering an effective amount of a compound described herein in combination with one or more other anti-retroviral agents to the subjection in need thereof.

Also provided is the use of a compound disclosed herein in the manufacture of a medicament for treating or combating HIV or retroviral infection or disease in a subject.

Further provided are processes for preparing a compound as described herein, wherein Y is N and Z is CH comprising heating an optionally substituted benzyloxymethylethyl ester in the presence of thiourea and iodomethane to form an optionally substituted 6-benzyl-3,4-dihydro-2-methylthiopyrimidin-4-one, and then reacting the optionally substituted 6-benzyl-3,4-dihydro-2-methylthiopyrimidin-4-one with a cyclic amine derivative compound to provide an optionally substituted 6-benzyl-3,4-dihydro-2-cyclic amino-pyrimidin-4-one product.

The oxo-pyrimidine compounds of the present invention belong to a class of anti-HIV agents that inhibit reverse transcriptase activity. These compounds can be assessed for their inhibitory ability in vitro by standard assays.

In still another embodiment, the active compound can be administered in combination or alternation with another anti-HIV agent. In combination therapy, effective dosages of two or more agents are administered together, whereas during alternation therapy an effective dosage of each agent is administered serially. The dosages depend on absorption, inactivation, and excretion rates of the drug as well as on other factors known to those of skill in the art. It is to be noted that dosage values will also vary with the severity of the condition to be alleviated. It is to be understood further that for any particular subject, specific dosage regimens and schedules should be adjusted over time according to individual need and the professional judgment of the person administering or supervising the administration of the compositions.

In a further embodiment of the present invention, an oxo-pyrimidine compound comprises the active agent, alone or in combination with another anti-viral agent, in a pharmaceutical composition that includes a pharmaceutically-acceptable carrier, excipient or diluent.

The invention provides novel compounds, compositions and methods for prophylactically inhibiting the spread of AIDS. The compounds of the present invention display excellent inhibition of HIV replication, and do so for a prolonged period of time, which renders them especially useful in prophylactic applications, wherein the frequency or duration of use are not always predictable. The compounds are also very useful in prophylactic applications because they deactivate the HIV virus upon contact at very low concentrations, before the virus has infected its host and begun replication. Non-nucleoside reverse transcriptase inhibitors (“NNRTI”) have proven especially invaluable in this type of application.

Thus, in one embodiment the invention provides a method for inhibiting sexual transmission of HIV comprising topically applying to the skin or epithelial tissue of a human a composition comprising a non-nucleoside reverse transcriptase inhibitor (“NNRTI”) that is able to inhibit viral replication for periods exceeding 12, 24 or even 36 days, at concentrations below even 10 μM.

In another embodiment the invention provides an oxo-pyrimidine compound of the present invention in the form of a cream, lotion, gel, or foam, comprising the oxo-pyrimidine compound.

In still another embodiment the invention provides a composition in the form of an intra-vaginal or intra-rectal pill or suppository comprising an oxo-pyrimidine compound of the present invention.

A still further embodiment provides a device for inhibiting the sexual transmission of HIV comprising: (a) a barrier structure for insertion into the vaginal cavity, and (b) a composition comprising an oxo-pyrimidine compound of the present invention.

Finally, the present invention includes one or more processes for preparing the optionally substituted oxo-pyrimidine compounds disclosed herein.

### BRIEF DESCRIPTION OF THE DRAWINGS

FIG. 1 illustrates a non-limiting example of a genus of oxo-pyrimidine compounds of the present invention useful in the treatment of HIV.

FIG. 2 illustrates non-limiting examples of particular of oxo-pyrimidine compounds of the present invention useful in the treatment of HIV.

FIG. 3 illustrates a non-limiting example of another genus of oxo-pyrimidine compounds of the present invention useful in the treatment of HIV.

FIG. 4 illustrates non-limiting examples of particular of oxo-pyrimidine compounds of the present invention useful in the treatment of HIV.

FIG. 5 is a non-limiting example of a synthesis for oxo-pyrimidine compounds.

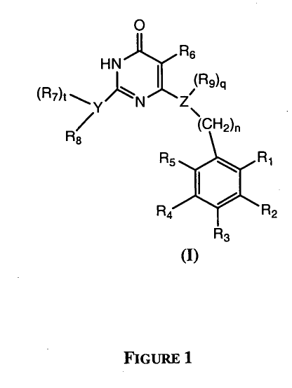

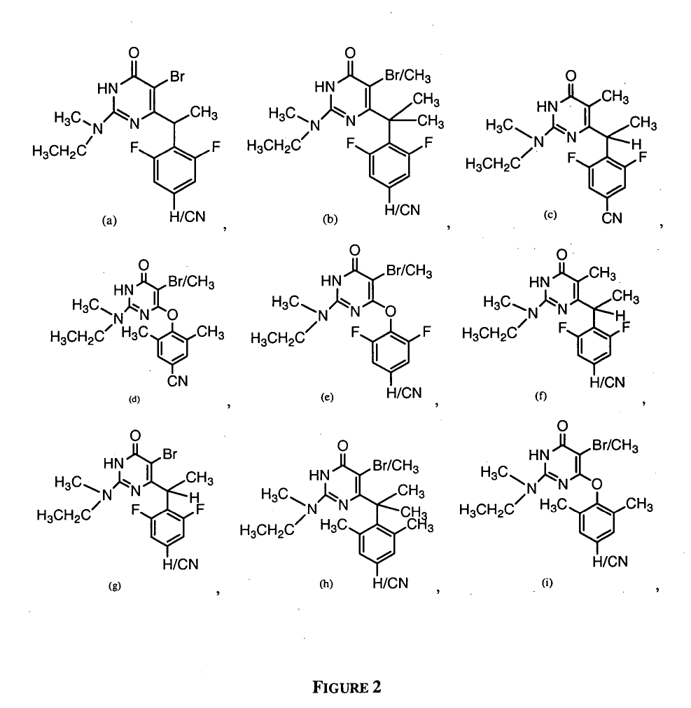

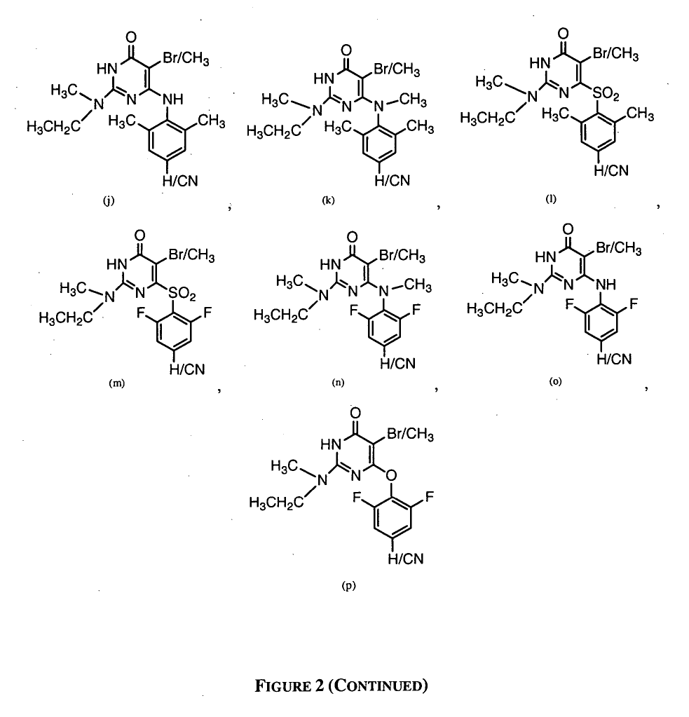

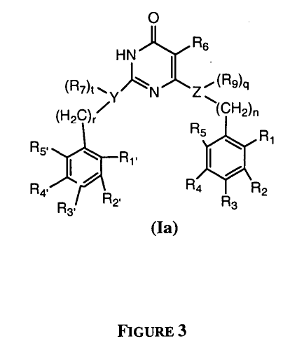

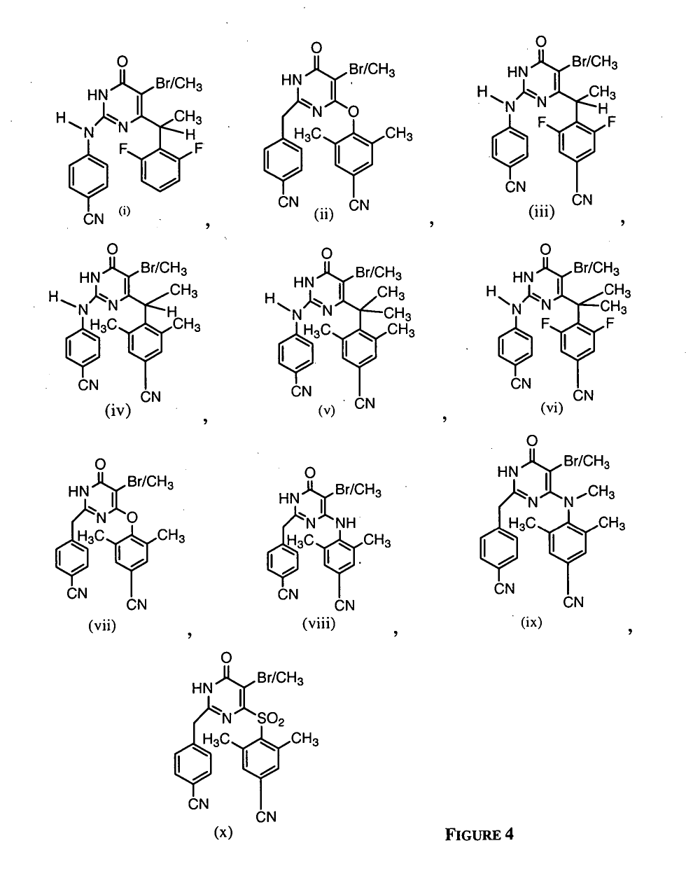

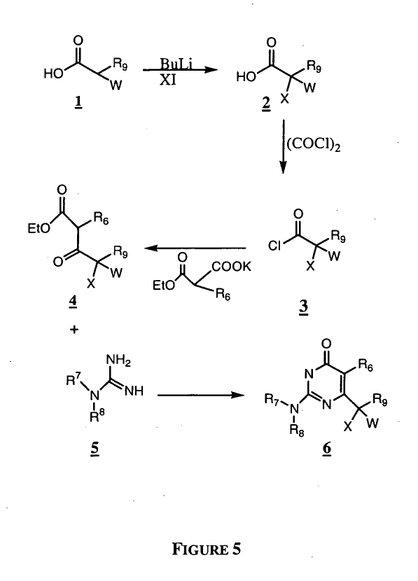

### DETAILED DESCRIPTION OF THE INVENTION

The invention disclosed herein is drawn to an oxo-pyrimidine compound, a pharmaceutically-acceptable salt, prodrug, stereoisomer or tautomer thereof, a process for preparing such a compound, a method of using the compound in a composition for the treatment of a subject, and in particular a human subject, infected with HIV, and the use of such a compound in the manufacture of a medicament. The present invention includes the administration of an effective HIV treatment amount of the oxo-pyrimidine compound as described herein, optionally with a pharmaceutically acceptable carrier, diluent or excipient.

The compounds of the present invention possess anti-viral and particularly anti-HIV activity, or are metabolized to a compound that exhibits such activity.

One or more of the following features characterize oxo-pyrimidine compounds, their pharmaceutically-acceptable salts, prodrugs, stereoisomers or tautomers of the present invention:
a) effective against HIV in a host;
b) effective against drug-resistant strains of HIV in a host;
c) effective against drug-resistant strains of UW due to a reverse transcriptase mutation, such as, for example, lysine 103→ asparagine and/or tyrosine 181→ cysteine in a host;
d) useful for the prophylaxis and/or treatment of an HIV infection within a host, especially in subjects diagnosed as having HIV infection or at risk for becoming infected with HIV;
e) useful in the manufacture of a medicament for the treatment or prophylaxis of an HIV infection within a host, especially in subjects diagnosed as having HIV infection or at risk for becoming infected with HIV;
f) useful for the treatment or prophylaxis in a subject of an HIV infection that is resistant to one or more reverse transcriptase inhibitors;
g) useful in the manufacture of a medicament for the treatment or prophylaxis of an HIV infection within a host wherein the infection is resistant to one or more reverse transcriptase inhibitors;
h) useful for the prophylaxis or treatment of an HIV infection as a form of salvage therapy in a host, especially in subjects diagnosed as having an HIV infection or at risk for becoming infected with HIV;
i) useful in the manufacture of a medicament for the treatment or prophylaxis of an HIV infection as a form of salvage therapy within a host, especially in subjects diagnosed as having an HIV infection or at risk for becoming infected with HIV;
j) useful for the treatment or prophylaxis of an HIV infection that is resistant to one or more reverse transcriptase inhibitors due to one or more reverse transcriptase mutations in a host, especially in subjects diagnosed as having an HIV infection or at risk for becoming infected with HIV;
k) useful in the manufacture of a medicament for the treatment or prophylaxis of an HIV infection that is resistant to one or more reverse transcriptase inhibitors due to one or more reverse transcriptase mutations in a host, especially in subjects diagnosed as having an HIV infection or at risk for becoming infected with HIV;
l) preparation by one or more distinct processes or syntheses as described herein;
m) preparation by one or more distinct process or synthesis whose product is a compound of the present invention substantially free from other chemical entities;
n) incorporation as active agent in a pharmaceutical composition that provides an effective anti-HIV treatment amount of the compound, optionally together with a pharmaceutically acceptable carrier, diluent or excipient;
o) incorporation as active agent in a pharmaceutical composition that provides an effective anti-HIV treatment amount of the compound, optionally together with a pharmaceutically acceptable carrier, diluent or excipient, and further optionally in combination with one or more other anti-HIV agents;
p) incorporation as active agent in a pharmaceutical composition that provides an effective anti-HIV treatment amount of the compound, wherein the HIV infection is resistant to one or more reverse transcriptase inhibitors, and optionally together with a pharmaceutically acceptable carrier, diluent or excipient;
q) incorporation as active agent in a pharmaceutical composition for the treatment or prophylaxis of an HIV infection in a host as a form of salvage therapy that provides an effective anti-HIV treatment amount of the compound, optionally together with a pharmaceutically acceptable carrier, diluent or excipient, and optionally in combination with one or more other anti-HIV agents;
r) incorporation as active agent in a pharmaceutical composition for the treatment or prophylaxis of an HIV infection that is resistant to one or more reverse transcriptase inhibitors, in a host and that provides an effective anti-HIV treatment amount of the compound, optionally together with a pharmaceutically acceptable carrier, diluent or excipient, and optionally in combination with one or more other anti-HIV agents;
s) useful for the treatment or prophylaxis of an HIV infection in a host that comprises administering to the host an anti-HIV effective treatment amount of a compound of the present invention, optionally in combination with a pharmaceutically-acceptable carrier, diluent or excipient;
t) useful for the treatment or prophylaxis of an HIV infection that is resistant to one or more reverse transcriptase inhibitors due to one or more reverse transcriptase mutations in a host, that comprises administering to the host an anti-HIV effective treatment amount of a compound of the present invention, optionally in combination with a pharmaceutically-acceptable carrier, diluent or excipient;
u) useful for the treatment or prophylaxis of an HIV infection in a host as a form of salvage therapy comprises administering to the host an anti-HIV effective treatment amount of a compound of the present invention, optionally in combination with a pharmaceutically-acceptable carrier, diluent or excipient, and optionally in combination and/or alternation with one or more other anti-HIV agents;
v) useful for the treatment or prophylaxis of an HIV infection that is resistant to one or more reverse transcriptase inhibitors due to one or more reverse transcriptase mutations in a host, that comprises administering to the host an anti-HIV effective treatment amount of a compound of the present invention, optionally in combination with a pharmaceutically-acceptable carrier, diluent or excipient, and optionally in combination and/or alternation with one or more other anti-HIV agents;
w) useful for the prophylaxis and/or treatment of an HIV infection within a host, optionally in combination with one or more other anti-HIV agents and optionally with a pharmaceutically-acceptable carrier, diluent or excipient;
x) useful for the prophylaxis and/or treatment of an HIV infection that is resistant to one or more reverse transcriptase inhibitors within a host, optionally in combination with one or more other anti-HIV agents and optionally with a pharmaceutically-acceptable carrier, diluent or excipient;
y) useful for the prophylaxis and/or treatment of an HIV infection within a host as a form of salvage therapy, optionally in combination with one or more other anti-HIV agents and optionally with a pharmaceutically-acceptable carrier, diluent or excipient;
z) useful for the prophylaxis and/or treatment of an HIV infection that is resistant to one or more reverse transcriptase inhibitors due to one or more reverse transcription mutations within a host, optionally in combination with one or more other anti-HIV agents and optionally with a pharmaceutically-acceptable carrier, diluent or excipient;
aa) useful in the manufacture of a medicament for the prophylaxis and/or treatment of an HIV infection within a host, optionally in combination with one or more other anti-HIV agents and optionally with a pharmaceutically-acceptable carrier, diluent or excipient;
bb) useful in the manufacture of a medicament for the prophylaxis and/or treatment of an HIV infection that is resistant to one or more reverse transcriptase inhibitors within a host, optionally in combination with one or more other anti-HIV agents and optionally with a pharmaceutically-acceptable carrier, diluent or excipient;
cc) useful in the manufacture of a medicament for the prophylaxis and/or treatment of an HIV infection within a host as a form of salvage therapy, optionally in combination with one or more other anti-HIV agents and optionally with a pharmaceutically-acceptable carrier, diluent or excipient; and
dd) useful in the manufacture of a medicament for the prophylaxis and/or treatment of an HIV infection that is resistant to one or more reverse transcriptase inhibitors due to one or more reverse transcriptase mutations within a host, optionally in combination with one or more other anti-HIV agents and optionally with a pharmaceutically-acceptable carrier, diluent or excipient.

In another aspect of the present invention, the active compound exhibits significant activity against drug-resistant forms of HIV, and thus exhibits cross-resistance against currently approved antiviral therapies. The term “significant activity against a drug resistant form of HIV” means that a compound (or its pharmaceutically-acceptable salt, prodrug, stereoisomeric or tautomeric form) is active against a mutant strain with an EC₅₀ against the mutant strain of less than approximately 50, 25, 10 or 1 μM concentration. In a preferred embodiment, the non-nucleoside reverse transcriptase inhibitors (NNRTIs) display an EC₅₀ (measured in molar concentrations) in a mutant HIV strain of less than about 5, 2.5, 1 or 0.1 μM concentration. In a non-limiting embodiment, one HIV mutant strain is a strain with a reverse transcriptase mutation at lysine 103→ asparagine and/or tyrosine 181→ cysteine.

In one aspect the invention provides a method for inhibiting sexual transmission of HIV comprising topically applying to the skin or epithelial tissue of a human a composition comprising a non-nucleoside reverse transcriptase inhibitor (“NNRTI”) that is able to inhibit viral replication for periods exceeding 12, 24, or even 36 days. In separate embodiments, the composition is able to inhibit viral replication for such prolonged periods at concentrations as low as 50, 35, 20, 10, or 5 μM. The ability of a compound to inhibit viral replication is preferably evaluated by the HIV-1 p24 antigen enzyme-linked immunosorbent assay. Suitable ELISA kits are available, for example, from Abbott Laboratories. Particular methods for their use are set forth in the examples herein. The ability of a non-nucleotide compound to inhibit reverse transcriptase can also be assessed by the methods set forth in the examples hereof.

The composition is preferably applied topically to any skin or epithelial tissue that comes in contact with bodily fluids of a sexual partner during sexual intercourse or foreplay, including the vaginal endothelium, the rectal endothelium, or the male genitalia. As used herein, the term “topical application” refers to something that is applied to and spread across the surface of the skin or a mucous membrane (by contrast, “systemic” administration refers to a drug or other compound that is ingested orally or injected beneath the skin). A condom lubricant or other genital lubricant is a topical agent as that term is used herein.

In one particular embodiment the NNRTI is an oxo-pyrimidine compound, as defined further herein.

Preferred compositions can take several forms. Thus, in one embodiment the composition is in the form of a cream, lotion, gel, or foam that is applied to the affected skin or epithelial cavity, and preferably spread over the entire skin or epithelial surface which is at risk of contact with bodily fluids. Such formulations, which are suitable for vaginal or rectal administration, may be present as aqueous or oily suspensions, solutions or emulsions (liquid formulations) containing in addition to the active ingredient, such carriers as are known in the art to be appropriate. For “stand-alone” lubricants (i.e., lubricants that are not pre-packaged with condoms), gels and similar aqueous formulations are generally preferred, for various reasons (both scientific and economic) known to those skilled in the art. These formulations are useful to protect not only against sexual transmission of HIV, but also to prevent infection of a baby during passage through the birth canal. Thus the vaginal administration can take place prior to sexual intercourse, during sexual intercourse, and immediately prior to childbirth.

One method of applying an anti-viral lubricant to the genitals, for the purposes disclosed herein, involves removing a small quantity (such as a teaspoon, or several milliliters) of a gel, cream, ointment, emulsion, or similar formulation from a plastic or metallic tube, jar, or similar container, or from a sealed plastic, metallic or other packet containing a single dose of such composition, and spreading the composition across the surface of the penis immediately before intercourse. Alternate methods of emplacement include: (1) spreading the composition upon accessible surfaces inside the vagina or rectum shortly before intercourse; and (2) emplacing a condom, diaphragm, or similar device, which has already been coated or otherwise contacted with an anti-viral lubricant, upon the penis or inside the vagina. In a preferred embodiment, any of these methods of spreading an anti-viral lubricant across the surfaces of the genitals causes the lubricant to coat and remain in contact with the genital and epithelial surfaces throughout intercourse.

In one embodiment the compositions are used in conjunction with condoms, to enhance the risk-reducing effectiveness of condoms and provide maximum protection for users. The composition can either be coated onto condoms during manufacture, and enclosed within conventional watertight plastic or foil packages that contain one condom per package, or it can be manually applied by a user to either the inside or the outside of a condom, immediately before use.

As used herein, “condom” refers to a barrier device which is used to provide a watertight physical barrier between male and female genitalia during sexual intercourse, and which is removed after intercourse. This term includes conventional condoms that cover the penis; it also includes so-called “female condoms” which are inserted into the vaginal cavity prior to intercourse. The term “condom” does not include diaphragms, cervical caps or other barrier devices that cover only a portion of the epithelial membranes inside the vaginal cavity. Preferably, condoms should be made of latex or a synthetic plastic material such as polyurethane, since these provide a high degree of protection against viruses.

In another embodiment the composition is in the form of an intra-vaginal pill, an intra-rectal pill, or a suppository. The suppository or pill should be inserted into the vaginal or rectal cavity in a manner that permits the suppository or pill, as it dissolves or erodes, to coat the vaginal or rectal walls with a prophylactic layer of the anti-HIV agent.

In still another embodiment the composition is topically applied by release from an intravaginal device. Devices such as vaginal rings, vaginal sponges, diaphrams, cervical caps, female condoms, and the like can be readily adapted to release the composition into the vaginal cavity after insertion.

Compositions used in the methods of this invention may also comprise other active agents, such as another agent to prevent HIV infection, and agents that protect individuals from conception and other sexually transmitted diseases. Thus, in another embodiment the compositions used in this invention further comprise a second anti-HIV agent, a virucide effective against viral infections other than HIV, and/or a spermicide.

In one particular embodiment, the composition contains nonoxynol, a widely-used spermicidal surfactant. The resulting composition could be regarded as a “bi-functional” composition, since it would have two active agents that provide two different desired functions, in a relatively inert carrier liquid; the nonoxynol would provide a spermicidal contraceptive agent, and the DABO would provide anti-viral properties. The nonoxynol is likely to cause some level of irritation, in at least some users; this is a regrettable but is a well-known side effect of spermicidal surfactants such as nonoxynol and octoxynol, which attack and destroy the lipid bilayer membranes that surround sperm cells and other mammalian cells.

The compositions used in this invention may also contain a lubricant that facilitates application of the composition to the desired areas of skin and epithelial tissue, and reduces friction during sexual intercourse. In the case of a pill or suppository, the lubricant can be applied to the exterior of the dosage form to facilitate insertion.

In still another embodiment the invention provides a device for inhibiting the sexual transmission of HiV comprising (a) a barrier structure for insertion into the vaginal cavity, and (b) a composition comprising a dihydro-alkyloxy-benzyl-oxopryimidine. As mentioned above, preferred devices which act as barrier structures, and which can be adapted to apply anti-HIV agent, include the vaginal sponge, diaphram, cervical cap, or condom (male or female).

The methods, compositions, and devices of this invention can be adapted generally to release active agent in a time sensitive manner that best corresponds to the timing of sexual activity. When topically applied as a lotion or gel, the compositions are preferably applied immediately prior to sexual activity. Other modes of application, such as devices and suppositories, can be designed to release active agent over a prolonged period of time, at a predetermined rate, depending upon the needs of the consumer.

Active Compounds of the Present Invention

Suitable oxo-pyrimnidine derivatives for practicing the present invention are represented by the Formula (I): 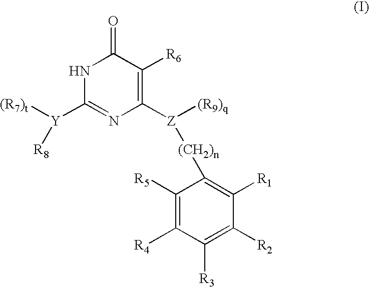
or a pharmaceutically acceptable salt, prodrug, N-oxide, quaternary amine, stereochemical isomer or tautomer thereof, wherein
a) each R₁, R₂, R₃, R₄R₅, independently, is —H; —C₁₋₆ alkyl; —C₂₋₆ alkenyl; —C₂₋₆ alkynyl; optionally substituted aryl; -aralkyl; -halo; -haloalkyl and —CF₃ in particular; —NO₂; —CN; —CONH₂; —CONH—C₁₋₆ alkyl; —CON(C₁₋₆ alkyl)₂; —NH₂; —NH—C₁₋₆ alkyl; —N(C₁₋₆ alkyl)₂; —NHC(O)-alkyl; —NHSO₂—C₁₋₆ alkyl; —SO₂NH₂; —SO₂NH—C₁₋₆ alkyl; —SO₂N(C₁₋₆ alkyl)₂; —SO₂NH-aryl; and —OW or —SW, where W is —H, or optionally substituted alkyl, aryl or aralkyl;
b) R₇ and R₈, each independently, is —H; —C₁₋₆ straight or branched chain alkyl; —C₂₋₆ alkenyl; —C₂₋₆ alkynyl; —C₃₋₆ cycloalkyl; 3-7 membered heterocycle; -aralkyl; -aryl; -haloalkyl; —CN; —CONH₂; —CONH—C₁₋₆ alkyl; —CON(C₁₋₆ alkyl)₂; —NH₂; —NH—C₁₋₆ alkyl; —N(C₁₋₆ alkyl)₂; —NHC(O)-C₁₋₆ alkyl; —NHSO₂—C₁₋₆ alkyl; —(CH₂)ₙ-phenyl; —SO₂NH₂; —SO₂NH—C₁₋₆ alkyl; —SO₂N(C₁₋₆ alkyl)₂; —SO₂NH-aryl; any of which may be substituted or unsubstituted; or
   R₇ and R₈, each independently, is —OW or —SW when Z is N or preferably C, and where W is —H or optionally substituted alkyl, aryl or aralkyl; or
   R₇ and R₉ together with the Y substituent to which they are attached, form an alicyclic, aryl or heterocyclic ring that optionally may have one or more O, S or N atoms, and further may be substituted or unsubstituted;
c) R₆ is —H; -halo; —C₁₋₆ straight or branched chain alkyl; —C₁₋₆ alkenyl; —C₁₋₆ alkynyl; —C₃₋₆ cycloalkyl; 3-7 membered heterocycle; -aryl; -aralkyl; -halo; -haloalkyl; —CN; —CONH₂; —CONH—C₁₋₆ alkyl; —CON(C₁₋₆ alkyl)₂; —NH₂; —NH—C₁₋₆ alkyl; —N(C₁₋₆ alkyl)₂; —NHC(O)—C₁₋₆ alkyl; —NHSO₂—C₁₋₆ alkyl; —SO₂NH₂; —SO₂NH—C₁₋₆ alkyl; —SO₂N(C₁₋₆ alkyl)₂; —SO₂NH-aryl; any of which may be substituted or unsubstituted; or
   R₆ is —OW or —SW when Z is N or preferably C, and where W is —H or optionally substituted alkyl, aryl or aralkyl;
d) R₉ is —H; —C₁₋₆ straight or branched chain alkyl; —C₁₋₆ alkenyl; —C₁₋₆ alkynyl; —C₃₋₆ cycloalkyl; 3-7 membered heterocycle; -aryl; -aralkyl; -halo; -haloalkyl; —CN; —CONH₂; —CONH—C₁₋₆ alkyl; —CON(C₁₋₆ alkyl)₂; —NH₂; —NH—C₁₋₆ alkyl; —N(C₁₋₆ alkyl)₂; —NHC(O)—C₁₋₆ alkyl; —NHSO₂—C₁₋₆ alkyl; —SO₂NH₂; —SO₂NH—C₁₋₆ alkyl; —SO₂N(C₁₋₆ alkyl)₂; —SO₂NH-aryl; any of which may be substituted or unsubstituted; or
   R₉ is —OW or —SW when Z is N or preferably C, and where W is —H or optionally substituted alkyl, aryl or aralkyl; or
e) alternatively, R₆ and R₉ taken together with the pyrimidinone ring and Z substituent to which they are attached respectively, form a bicyclic heterocycle that optionally may have additional O, S or N atoms, and further may be substituted or unsubstituted; and
   where t is 1, 2, or 3, then each occurrence of R₇ is independently selected from the substituents listed herein for R₇;
   where q is 1, 2 or 3, then each occurrence of R₉ is independently selected from the substituents listed herein for R₉;
f) Y is O, S, or SO₂, and t is 0; or
   Y is CH or N, and t is 1; or
   Y is C or N⁺ (with a suitable counter ion), and t is 2;
g) Z is O, S, or SO₂, and q is 0; or
   Z is CH or N, and q is 1; or
   Z is C or N⁺ (with a suitable counter ion), and q is 2; and
h) n is 0-4.

In one preferred embodiment of Formula (I), R₆ is bromo or methyl, Z is CH, R₉ is methyl, R₁ and R₅ are both fluoro, R₃ is H or cyano, Y is N, R₇ is methyl, R₈ is ethyl, t=1,and q=1.

In a second preferred embodiment, R₆ is bromo or methyl, Z is O, R₁ and R₅ are both fluoro, R₃ is H or cyano, Y is N, R₇ is methyl, R₈ is ethyl, t=1, and q=0.

In another preferred embodiment, R₆ is bromo or methyl, Z is CH, R₉ is methyl, R₁ and R₅ are both fluoro, R₃ is H or cyano, Y is N, R₇ is methyl, R₈ is -p-cyano-phenyl, t=1, and q=1.

In yet another preferred embodiment, R₆ is bromo or methyl, Z is O, R₁ and R₅ are both methyl, R₃ is H or cyano, Y is CH, R₇ is H, R₈ is -p-cyano-phenyl, t=1, and q=0.

In still another preferred embodiment, R₆ is bromo or methyl, Z is CH, R₉ is methyl, R₁ and R₅ are both fluoro, R₃ is cyano, Y is N, R₇ is H, R₈ is -p-cyano-phenyl, t=1, and q=1.

In another preferred embodiment, R₆ is bromo or methyl, Z is CH, R₉ is methyl, R₁ and R₅ are both methyl, R₃ is cyano, Y is N, R₇ is H, R₈ is -p-cyano-phenyl, t=1, and q=1.

In still another preferred embodiment, R₆ is bromo or methyl, Z is CH, R₉ is methyl, R₁ and R₅ are both methyl, R₃ is cyano, Y is N, R₇ is H, R₈ is -p-cyano-phenyl, t=1,and q=1.

In yet another preferred embodiment, R₆ is bromo or methyl, Z is CH, R₉ is methyl, R₁ and R₅ are both fluoro, R₃ is H or cyano, Y is N, R₇ is methyl, R₈ is -p-cyano-phenyl, t=1, and q=1.

In still another preferred embodiment, R₆ is methyl, Z is CH, R₉ is methyl, R₁ and R₅ are both fluoro, R₃ is cyano, Y is N, R₇ is methyl, R₈ is ethyl, t=1, and q=1.

In yet another preferred embodiment, R₆ is bromo, Z is CH, R₉ is methyl, R₁ and R₅ are both fluoro, R₃ is H or cyano, Y is N, R₇ is methyl, R₈ is ethyl, t=1, and q=1.

In still yet another preferred embodiment, R₆ is bromo or methyl, Z is C, R₉ is dimethyl, R₁ and R₅ are both fluoro, R₃ is H or cyano, Y is N, R₇ is methyl, R₈ is ethyl, t=1, and q=1.

In still another preferred embodiment, R₆ is bromo or methyl, Z is C, R₉ is dimethyl, R₁ and R₅ are both methyl, R₃ is H or cyano, Y is N, R₇ is methyl, R₈ is ethyl, t=1,and q=1.

In yet another preferred embodiment, R₆ is bromo or methyl, Z is O, R₁ and R₅ are both methyl, R₃ is cyano, Y is N, R₇ is methyl, R₈ is ethyl, t=1, and q=0.

In still another preferred embodiment, R₆ is bromo or methyl, Z is N, R₉ is H, R₁ and R₅ are both methyl, R₃ is cyano, Y is N, R₇ is methyl, R₈ is ethyl, t=1, and q=1.

In yet still another preferred embodiment, R₆ is bromo or methyl, Z is N, R₉ is methyl, R₁ and R₅ are both methyl, R₃ is cyano, Y is N, R₇ is methyl, R₈ is ethyl, t=1, and q=1.

In still another preferred embodiment, R₆ is bromo or methyl, Z is SO₂, R₁ and R₅ are both methyl, R₃ is cyano, Y is N, R₇ is methyl, R₈ is ethyl, t=1, and q=0.

In yet another preferred embodiment, R₆ is bromo or methyl, Z is O, R₁ and R₅ are both fluoro, R₃ is H or cyano, Y is N, R₇ is methyl, R₈ is ethyl, t=1, and q=0.

In still yet another preferred embodiment, R₆ is bromo or methyl, Z is N, R₉ is H, R₁ and R₅ are both fluoro, R₃ is H or cyano, Y is N, R₇ is methyl, R₈ is ethyl, t=1, and q=1.

In still yet another preferred embodiment, R₆ is bromo or methyl, Z is N, R₉ is methyl, R₁ and R₅ are both fluoro, R₃ is H or cyano, Y is N, R₇ is methyl, R₈ is ethyl, t=1,and q=1.

In another preferred embodiment, R₆ is bromo or methyl, Z is SO₂, R₁ and R₅ are both fluoro, R₃ is H or cyano, Y is N, R₇ is methyl, R₈ is ethyl, t=1, and q=0.

In yet another preferred embodiment, R₆ is bromo or methyl, Z is C, R₉ is dimethyl, R₁ and R₅ are both fluoro, R₃ is H or cyano, Y is N, R₇ is methyl, R₈ is ethyl, t=1,and q=2.

In still other preferred embodiments, R₈ is optionally substituted —(CH₂)ᵣ-phenyl and the compounds of the present invention are represented by the Formula (Ia): 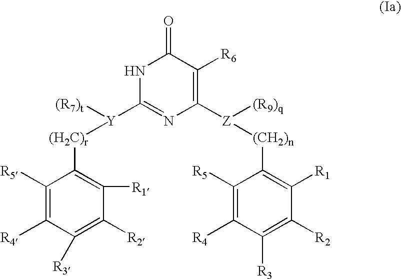
or a pharmaceutically acceptable salt, prodrug, N-oxide, quaternary amine, stereochemical isomer or tautomer thereof, wherein
a) each R₁, R₂, R₃, R₄R₅, R_{1′}, R_{2′}, R_{3′}, R_{4′} and R_{5′} independently, is —H; —C₁₋₆ alkyl; —C₂₋₆ alkenyl; —C₂₋₆ alkynyl; -aralkyl; optionally substituted aryl; -halo; -haloalkyl and —CF₃ in particular; —NO₂; —CN; —CONH₂; —CONH—C₁₋₆ alkyl; —CON(C₁₋₆ alkyl)₂; —NH₂; —NH—C₁₋₆ alkyl; —N(C₁₋₆ alkyl)₂; —NHC(O)-alkyl; —NHSO₂—C₁₋₆ alkyl; —SO₂NH₂; —SO₂NH—C₁₋₆alkyl; —SO₂N(C₁₋₆ alkyl)₂; —SO₂NH-aryl; and —OW or —SW, where W is —H, or optionally substituted alkyl, aryl or aralkyl;
b) R₆ and R₇, each independently, is H; C₁₋₆ straight or branched chain alkyl; —C₂₋₆ alkenyl; —C₂₋₆ alkynyl; aralkyl; halo; haloalkyl and CF₃ in particular; C₃₋₆ cycloalkyl; 3-7 membered heterocycle; or -aryl; —NO₂; —CN; —CONH₂; —CONH—C₁₋₆ alkyl; —CON(C₁₋₆ alkyl)₂; —NH₂; —NH—C₁₋₆ alkyl; —N(C₁₋₆ alkyl)₂; —NHC(O)—C₁₋₆ alkyl; —NHSO₂—C₁₋₆ alkyl; —SO₂NH₂; —SO₂NH—C₁₋₆ alkyl; —SO₂N(C₁₋₆ alkyl)₂; —SO₂NH-aryl; —(CH₂)ₙ-phenyl; any of which may be substituted or unsubstituted; or
   R₆ and R₇, each independently, is —OW or —SW when Z is N or preferably C, and where W is —H or optionally substituted alkyl, aryl or aralkyl;
c) R₉ is —H; —C₁₋₆ straight or branched chain alkyl; —C₁₋₆ alkenyl; —C₁₋₆ alkynyl; —C₃₋₆ cycloalkyl; 3-7 membered heterocycle; -aryl; -aralkyl; -halo; -haloalkyl; —CN; —CONH₂; —CONH—C₁₋₆alkyl; —CON(C₁₋₆ alkyl)₂; —NH₂; —NH—C₁₋₆ alkyl; —N(C₁₋₆ alkyl)₂; —NHC(O)—C₁₋₆ alkyl; —NHSO₂—C₁₋₆ alkyl; —SO₂NH₂; —SO₂NH—C₁₋₆ alkyl; —SO₂N(C₁₋₆ alkyl)₂; —SO₂NH-aryl; any of which may be substituted or unsubstituted; or
d) R₉ is —OW or —SW when Z is N or preferably C, and where W is —H or optionally substituted alkyl, aryl or aralkyl; or
e) alternatively, R₆ and R₉ taken together with the pyrimidinone ring and Z substituent to which they are attached, form a bicyclic heterocycle that optionally may have additional O, S or N atoms, and which further may be substituted or unsubstituted; and
   where t is 1, 2, or 3, then each occurrence of R₇ is independently selected from the substituents listed herein for R₇;
   where q is 1, 2 or 3, then each occurrence of R₉ is independently selected from the substituents listed herein for R₉;
e) r is 0-4;
f) Y is O, S, or SO₂, and t is 0; or
   Y is CH or N, and t is 1; or
   Y is C or N⁺ (with a suitable counter ion), and t is 2;
g) Z is O, S, or SO₂, and q is 0; or
   Z is CH or N, and q is 1; or
   Z is C or N⁺ (with a suitable counter ion), and q is 2; and
h) n is 0-4.

Preferred embodiments of Formula (Ia) include the following:

In one preferred embodiment, Y is N, R₇ is H, Z is CH, R₆ is bromo or methyl, R₉ is methyl, R₁ and R₅ are both F and R_{1′} and R_{5′} are both H, or R₁ and R₅ are both H and R_{1′} and R_{5′} are both F, R₃ and R_{3′} are both cyano, R₂, R_{2′}, R₄ and R_{4′} are all H, t=1, q=1, r=0 or 1, and n=0 or 1.

In another preferred embodiment, Y is N, R₇ is H, Z is CH, R₆ is bromo or methyl, R₉ is methyl, R₁ and R₅ are each methyl, R₃ and R_{3′} are both cyano, R_{1′}, R_{5′}, R₂, R_{2′}, R₄ and R_{4′} are all H,, t=1, q=1, r=0 or 1, and n=0 or 1.

In yet another preferred embodiment, Y is N, R₇ is H, Z is CH, R₆ is bromo or methyl, R₉ is methyl, R_{1 and R}₅ are each methyl, R₃ is H, R_{3′} is cyano, R_{1′}, R_{5′}, R₂, R_{2′}, R₄ and R_{4′} are all H, , t=1, q=1, r=0 or 1, and n=0 or 1.

In another preferred embodiment, Y is N, R₇ is H, Z is C, R₆ is bromo or methyl, R₉ is dimethyl, R₁ and R₅ are each methyl, R₃ and R_{3′} are each H or cyano, R_{1′}, R_{5′}, R₂, R_{2′}, R₄ and R_{4′} are all H, , t=1, q=2, r=0 or 1, and n=0 or 1.

In yet still another preferred embodiment, Y is N, R₇ is H, Z is C, R₆ is bromo or methyl, R₉ is dimethyl, R₁ and R₅ are each fluoro, R₃ and R_{3′} are each H or cyano, R_{1′}, R_{5′}, R₂,R_{2′}, R₄ and R_{4′} are all H, , t=1, q=2, r=0 or 1, and n=0 or 1.

In still another preferred embodiment, Y is CH, Z is O, R₆ is bromo or methyl, R₁ and R₅ are each methyl, R₃ and R_{3′} are each cyano, R₇ is H, R_{1′}, R_{5′}, R₂, R_{2′}, R₄ and R_{4′} are all H, t=1, q=0, r=0 or 1, and n=0 or 1.

In yet another preferred embodiment, Y is CH, Z is N, R₉ is H, R₆ is bromo or methyl, R₇ is H, R₁ and R₅ are each methyl, R₃ and R_{3′} are each cyano, R_{1′}, R_{5′}, R₂, R_{2′}, R₄ and R_{4′} are all H, t=1, q=1, r=0 or 1, and n=0 or 1.

In still another preferred embodiment, Y is CH, Z is N, R₉ is methyl, R₆ is bromo or methyl, R₇ is methyl, R₁ and R₅ are each methyl, R₃ and R₃ are each cyano, R_{1′}, R_{5′}, R₂,R_{2′}, R₄ and R_{4′} are all H, , t=1, q=1, r=0 or 1, and n=0 or 1.

In a final preferred embodiment, Y is CH, Z is SO₂, R₆ is bromo or methyl, R₇ is H, R₁ and R₅ are each methyl, R₃ and R_{3′} each cyano, and R_{1′}, R_{5′}, R₂, R_{2′}, R₄ and R_{4′} are all H, , t=1, q=0, r=0 or 1, and n=0 or 1.

In one particular embodiment, the oxo-pyrimidine compound is a compound having the following structure: 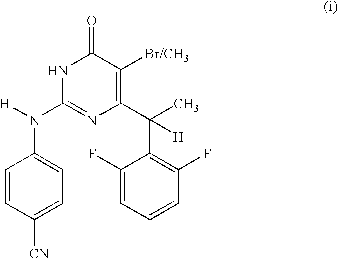
or a pharmaceutically-acceptable salt, prodrug, stereoisomer or tautomer thereof, including chiral forms where a chiral center is present.

In another particular embodiment, the oxo-pyrimidine compound is a compound of the structure: 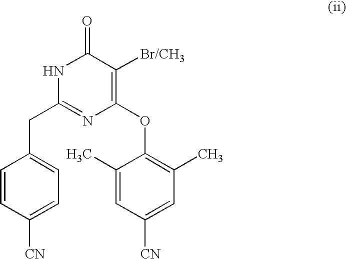
or a pharmaceutically-acceptable salt, prodrug, stereoisomer or tautomer thereof.

In yet another particular embodiment, the oxo-pyrimidine comopund is a compound that has the following structure: 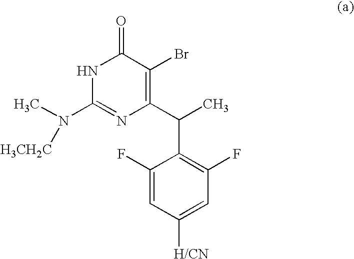
or a pharmaceutically-acceptable salt, prodrug, stereoisomer or tautomer thereof.

In still another particular embodiment of the present invention, the oxo-pyrimidine compound is a compound of the following structure: 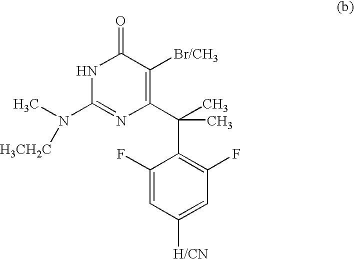
or a pharmaceutically-acceptable salt, prodrug, stereoisomer or tautomer thereof.

In yet another particular embodiment of the present invention, the oxo-pyrimidine compound has the following structure: 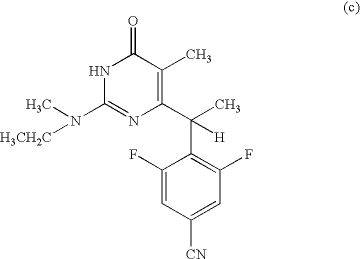
or a pharmaceutically-acceptable salt, prodrug, stereoisomer or tautomer thereof.

In still yet another particular embodiment of the present invention, the oxo-pyrimidine compound has the following structure: 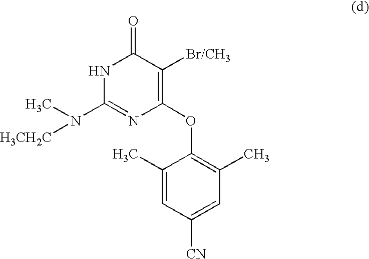
or a pharmaceutically-acceptable salt, prodrug, stereoisomer or tautomer thereof.

In yet another particular embodiment, the oxo-pyrimidine compound is a compound that has the following structure: 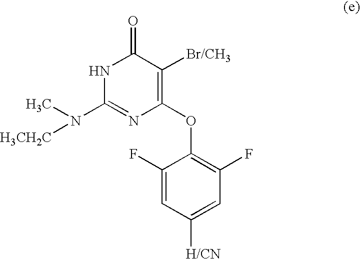
or a pharmaceutically-acceptable salt, prodrug, stereoisomer or tautomer thereof.

In still another particular embodiment, the oxo-pyrimidine compound is a compound that has the following structure: 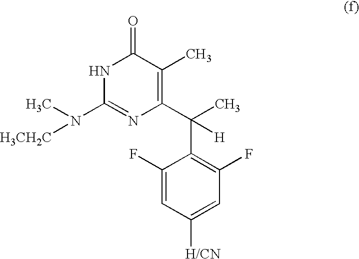
or a pharmaceutically-acceptable salt, prodrug, stereoisomer or tautomer thereof.

Each embodiment of the present invention, including those presented above, includes chiral forms of the compound where a chiral center is present.

The oxo-pyrimidine compound of this invention belong to a class of anti-HIV agents that inhibit HIV reverse transcriptase activity. Compounds are screened for their ability to inhibit HIV reverse transcriptase activity in vitro according to screening methods set forth more particularly below. The spectrum of activity exhibited by any particular compound is determined by evaluating the compound in assays described earlier in this specification or with other confirmatory assays known to those skilled in the art of anti-HIV compounds. Preferred compounds exhibit an EC₅₀ of less than 10-15 μM.

An active compound may be administered as a salt or prodrug that, upon administration to the recipient, is capable of providing directly or indirectly the parent compound, or that exhibits activity itself. Nonlimiting examples include a pharmaceutically-acceptable salt, alternatively referred to as a “physiologically-acceptable salt”. In addition, modifications made to a compound can affect its biologic activity, in some cases increasing the activity over the parent compound. This activity can be assessed by preparing a salt or prodrug form of the compound, and testing its antiviral activity by using methods described herein or other methods known to those of skill in the art of NNRTIs.

Definitions

The following definitions and term construction are intended, unless otherwise indicated.

Ranges, specific values, and preferred values listed for radicals, substituents and derivatives are for illustration only, and do not exclude other defined values or values within defined ranges for the radicals, substituents and derivatives.

“Halo” iincludes fluoro, chloro, bromo or iodo.

“Alkyl”, “alkoxy”, “alkenyl”, “alkynyl”, etc., includes both straight chain and branched groups. However, reference to an individual radical such as “propyl” embraces only that straight-chain radical, whereas a branched chain isomer such as “isopropyl” is specifically termed such.

“Alkyl” as used herein and unless otherwise specified, includes a saturated, straight, branched, or cyclic, primary, secondary, or tertiary hydrocarbon of C₁₋₁₀, and specifically includes methyl, ethyl, propyl, isopropyl, cyclopropyl, butyl, isobutyl, t-butyl, pentyl, cyclopentyl, isopentyl, neopentyl, hexyl, isohexyl, cyclohexyl, cyclohexylmethyl, 3-methylpentyl, 2,2-dimethylbutyl, and 2,3-dimethylbutyl. When the context of this document allows alkyl to be substituted, the moieties with which the alkyl group may be substituted include but not limited to hydroxyl, amino, alkylamino, arylamino, alkoxy, aryloxy, aryl, heterocyclyl, halo, carboxy, acyl, acyloxy, amido, nitro, cyano, sulfonic acid, sulfate, phosphonic acid, phosphate, or phosphonate, either protected or unprotected as needed, as known to those skilled in the art and as taught, for example, in Greene et al., Protective Groups in Organic Synthesis, John Wiley and Sons, Third Ed., 1999.

The term “lower alkyl” as used herein and unless otherwise specified, includes a C₁₋₄ saturated, straight, branched, or if appropriate, cyclic (for example, cyclopropyl) alkyl group, including both substituted and unsubstituted forms. Unless otherwise specifically stated in this application, when alkyl is a suitable moiety, lower alkyl is preferred. Similarly, when alkyl or lower alkyl is a suitable moiety, unsubstituted alkyl or lower alkyl is preferred.

The terms “alkenyl” and “alkynyl” refer to alkyl moieties, including both substituted and unsubstituted forms wherein at least one saturated C—C bond is replaced by a double or triple bond. Thus, C₂₋₆ alkenyl may be vinyl, allyl, 1-propenyl, 2-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, or 5-hexenyl. Similarly, C₂₋₆ alkynyl may be ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, or 5-hexynyl.

The term “alkylene” includes a saturated, straight chain, divalent alkyl radical of the formula —(CH₂)ₙ—, wherein “n” may be any whole integer from 1 to 10.

As used herein with exceptions as noted, “aryl” is intended to mean any stable monocyclic, bicyclic or tricyclic carbon ring of up to 8 members in each ring, wherein at least one ring is aromatic as defined by the Huckel 4n+2 rule. Examples of aryl ring systems include phenyl, naphthyl, tetrahydronaphthyl, and biphenyl. The aryl group may be substituted with one or more moieties including but not limited to hydroxyl, amino, alkylamino, arylamino, alkoxy, aryloxy, alkyl, heterocyclyl, halo, carboxy, acyl, acyloxy, amido, nitro, cyano, sulfonamido, sulfonic acid, sulfate, phosphonic acid, phosphate, or phosphonate, either protected or unprotected as needed, as known to those skilled in the art and as taught, for example, in Greene et al., Protective Groups in Organic Synthesis, John Wiley and Sons, Third Ed., 1999.

The term “heterocycle” or “heterocyclic” as used herein except where noted, includes a stable 5- to 7-membered monocyclic or stable 8- to 11-membered bicyclic heterocyclic ring which is either saturated or unsaturated, including heteroaryl, and which consists of carbon atoms and from one to three heteroatoms including but not limited to O, S, N and P; and wherein the nitrogen and sulfur heteroatoms may optionally be oxidized, and/or the nitrogen heteroatom quaternized, and including any bicyclic group in which any of the above-identified heterocyclic rings is fused to a benzene ring. The heterocyclic ring may be attached at any heteroatom or carbon atom which results in the creation of a stable structure. The heteroaromatic ring may be partially or totally hydrogenated, as desired. For example, dihydropyridine may be used in place of pyridine. Functional oxygen and nitrogen groups on a heteroaryl may be protected as necessary or desired. Suitable protecting groups for oxygen or nitrogen include trimethylsilyl, dimethylhexylsilyl, t-butyldimethylsilyl, t-butyldiphenylsilyl, trityl, substituted trityl, alkyl, methanesulfonyl, p-toluenesulfonyl, or acyl groups such as acetyl and propionyl.

Non-limiting examples of heteroaryl and heterocyclic groups include furyl, pyridyl, pyrimidyl, piperidinyl, piperazinyl, thienyl, pyrrolyl, pyrrolinyl, pyrrolidinyl, imidazolyl, tetrazolyl, triazolyl, triazinyl, thiazinyl, oxazolyl, purinyl, carbazolyl, quinolinyl, pyrazolyl, morpholinyl, benzimidazolyl, and the like. Any of the heteroaromatic and heterocyclic moieties may be optionally substituted as described above for aryl, including substitution(s) with one or more hydroyxl, amino, alkylamino, arylamino, alkoxy, aryloxy, alkyl, heterocyclyl, halo, carboxy, acyl, acyloxy, amido, nitro, cyano, sulfonic acid, sulfate, phosphonic acid, phosphate, or phosphonate, either protected or unprotected as needed, as known to those skilled in the art and as taught, for example, in Greene et al., Protective Groups in Organic Synthesis, John Wiley and Sons, Third Ed., 1999.

The term “acyl” includes a compound of the formula “RC(O)—”, wherein R is substituted or unsubstituted alkyl or aryl as defined herein.

The term “carboxyl” includes a compound of the formula “RCOOH”, wherein R is substituted or unsubstituted alkyl or aryl as defined herein.

The term “aralkyl” as used herein unless otherwise specified, includes an aryl group as defined above linked to the molecule through an alkyl group as defined above.

The term “alkaryl” as used herein unless otherwise specified, includes an alkyl group as defined above linked to the molecule through an aryl group as defined above.

The term “alkoxy” as used herein unless otherwise specified, includes a moiety of the structure “—O—alkyl”, where alkyl is as defined above.

The term “amino” as used herein unless otherwise specified, includes a moiety represented by the structure “—NR₂”, and includes primary amines, and secondary and tertiary amines optionally substituted by alkyl, aryl, heterocyclyl, and/or sulfonyl groups. Thus, R₂ may represent two hydrogens, two alkyl moieties, or one hydrogen and one alkyl moiety.

The term “amido” as used herein unless otherwise specified, includes a moiety represented by the structure “—C(O)NR₂”, wherein R is an H, alkyl, aryl, acyl, heterocyclyl and/or a sulfonyl group.

As used herein, an “amino acid” is a natural amino acid residue (i.e., Ala, Arg, Asn, Asp, Cys, Glu, Gln, Gly, His, Hyl, Hyp, Ile, Leu, Lys, Met, Phe, Pro, Ser, Thr, Trp, Tyr and Val) in D or L form, or an unnatural amino acid residue having one or more open valences such as, for example, t-butylglycine, omithine, hippuric acid and phosphothreonine. Other unnatural amino acids are those represented by the formula “NH₂(CH₂),COOH”, wherein y is 2-12, and includes aminoalkanoic acids such as ε-amino caproic acid (H₂N—(CH₂)₅—COOH). The term also comprises natural and unnatural amino acids bearing amino-protecting groups such as acyl, trifluoroacetyl and benzyloxycarbonyl, as well as natural and unnatural aminot acids protected at carboxy moieties by protecting groups such as C₁₋₆ alkyl, phenyl or benzyl ester and amide, and protecting groups known to those of skill in the art.

The term “quaternary amine” as used herein includes quaternary ammonium salts that have a positively charged nitrogen. They are formed by the reaction between a basic nitrogen in the compound of interest and an appropriate quaternizing agent such as, for example, methyliodide or benzyliodide. Appropriate counterions accompanying a quaternary amine include acetate, trifluoroacetate, chloro and bromo ions.

As used herein, the term “N-oxides” denotes a state of the compounds of the present invention in which one or more nitrogen atoms are oxidized.

As used herein, a “retrovirus” includes any virus that expresses reverse transcriptase. Examples of a retrovirus include but are not limited to, HIV-1, HIV-2, HTLV-I, HTLV-II, FeLV, FIV, SIV, AMV, MMTV, and MoMuLV.

As used herein, “reverse transcriptase” or “RT” refers to an enzyme having a non-nucleoside inhibitory binding site similar to that of HIV-1 RT, and to which ligands, which bind the composite binding pocket of the compounds of the present invention, also will bind. One measure of RT activity is viral replication. A measure of HIV-1 viral replication is the automated assay that utilizes MTT, as described earlier in this specification. Another measure is the p24 core antigen enzyme immunoassay, such as, for example, the assay commercially available from Coulter Corporation Immunotech, Inc.® (Westbrook, Mich.). Another means for measuring RT activity is by assaying recombinant HIV-1 reverse transcriptase activity, such as, for example, by using the Quan-T-RT™ assay system commercially available from Amersham® (Arlington Heights, Ill.) and as described by Bosworth et al., Nature, 1989, 341:167-168.

As used herein, a compound that “inhibits replication of human immunodeficiency virus (HIV)” means a compound that, when contacted with HIV-1, for example, via HIV-infected cells, effects a reduction in the amount of HIV-1 as compared with an untreated control. Inhibition of replication of HIV-1 may be measured by any means known to those skilled in the art, such as, for example, by the p24 assay disclosed above.

The reagent denoted “mCPBA” in the synthesis schemes is meta-chloro-peroxybenzoic acid.

The term “salvage therapy” as used herein means a compound that can be taken with any regimen after a patient's initial treatment regimen has failed.

As used herein, the term “host” refers to a multicellular or unicellular organism in which the virus can replicate. Thus, “host” includes a cell line, an mammal and, preferably, a human. Alternatively, a host can be carrying a part of the HIV genome whose replication or function may be altered by the compounds of the present invention. The term host specifically refers to infected cells, cells transfected with all or part of the HIV genome, and mammals, especially primates including chimpanzees and humans. In most mammal applications of the present invention, the host is a human patient. Veterinary applications, however, are clearly anticipated by the present invention, such as, for example, in chimpanzees.

Pharmaceutically Acceptable Salts, Prodrugs, Stereoisomers and Tautomers

The terms “stereoisomer” and “tautomer” as used herein include all possible stereoisomeric and tautomeric forms of the compounds of the present invention, as well as their quaternary amine, salt, solvate, prodrug, derivative, and N-oxide forms. Where the compounds of the general formulae (I) and (II) contain one or more chiral centers, all possible enantiomeric and diastereomeric forms are included.

The term “pharmaceutically acceptable salt” refers to the state of a compound in which the compound carries a counterion that is pharmaceutically acceptable. Such salts are non-toxic, therapeutically useful forms of the compounds of the present invention. Any salt that retains the desired biological activity of the compounds contained herein and that exhibits minimal or no undesired or toxicological effects is intended for inclusion here. Pharmaceutically acceptable salts include those derived from pharmaceutically acceptable organic or inorganic acids and bases. Non-pharmaceutically acceptable acids and bases also find use herein, as for example, in the synthesis and/or purification of the compounds of interest. Thus, all “salts” are intended for inclusion here.

Non-limiting examples of suitable salts include those derived from inorganic acids, such as, for example, hydrochloric acid, hydrobromic acid, sulfuric acid, phosphoric acid, nitric acid, bicarbonic acid, carbonic acid; and salts formed with organic acids, such as, for example, formic acid, acetic acid, oxalic acid, tartaric acid, succinic acid, malic acid, malonic acid, ascorbic acid, citric acid, benzoic acid, tannic acid, palmoic acid, alginic acid, polyglutamic acid, tosic acid, methanesulfonic acid, naphthalenesulfonic acid, naphthalenedisulfonic acid, α-ketoglutaric acid, α-glycerophosphoric acid and polygalacturonic acid. Suitable salts include those derived from alkali metals such as lithium, potassium and sodium, from alkaline earth metals such as calcium and magnesium, as well as from other acids well known to those of skill in the pharmaceutical art. Other suitable salts include those derived from metal cations such as zinc, bismuth, barium, or aluminum, or with a cation formed from an amine, such as ammonia, NN-dibenzylethylene-diamine, D-glucosamine,tetraethylammonium, or ethylenediamine. Moreover, suitable salts include those derived from a combination of acids and bases, such as, for example, a zinc tannate salt.

A pharmaceutically acceptable prodrug refers to a compound that is metabolized (i.e., hydrolyzed or oxidized, for example) in the host to form a compound of the present invention. Typical examples of prodrugs include compounds that have biologically labile protecting groups on a functional moiety of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, and/or dephosphorylated to produce the active compound.

The compounds of the present invention either possess antiviral activity against retroviruses and HIV in particular, or are metabolized to a compound that exhibits such activity.

Any of the oxo-pyrimidine compounds described herein may be administered as a prodrug to increase the activity, bioavailability, stability, or otherwise alter the properties of the oxo-pyrimidine. A number of prodrug ligands also are known. In general, acylation, alkylation or other lipophilic modifications of a heteroatom of the oxo-pyrimidine will increase the stability of the compound. Examples of substituent groups that can replace one or more hydrogens on a heterocycle include, but are not limited to, alkyl, aryl, steroidal, carbohydrate including sugars, 1,2-diacylglycerol, phospholipid, phosphotidylcholine, phosphocholine, and/or alcohol. Any of these may be used in combination with the disclosed oxo-pyrimidine compound to achieve a desired effect.

Combination or Alternation Therapy

In a certain embodiments, the oxo-pyrimidine compound of the present invention is administered in combination and/or alternation with one or more other anti-retroviral or anti-HIV agent. In one embodiment, the effect of administering two or more such agents in combination and/or alternation produces a synergistic effect in inhibiting HIV replication. In another embodiment, the effect of administering two or more such agents in combination and/or alternation produces an additive effect in inhibiting HIV replication.

Drug resistance most typically occurs by mutation of a gene that encodes for an enzyme used in the viral replication cycle, and most typically in the case of HIV, in either the reverse transcriptase or protease genes. It has been demonstrated that the efficacy of an anti-HIV drug can be prolonged, augmented or restored by administering the compound in combination or alternation with a second, and perhaps third, antiviral compound that induces a different mutation(s) from that selected for by the principle drug. Such drug combinations simultaneously reduce the possibility of resistance to any single drug and any associated toxic effects. Alternatively, the pharmacokinetics, biodistribution, or other parameters of the drug can be altered by such combination or alternation therapy. For example, the use of a combination of drugs may permit an individual drug within that combination to be given at a dosage lower than what would be required when the drug is administered as a monotherapeutic. Likewise, when drugs that target different stages of the viral life cycle are combined, there exists the possibility for potentiating their effects. Moreover, use of combinations of drugs could lower or eliminate undesirable side-effects from a single drug while still producing anti-viral activity. In general, combination therapy is typically preferred over alternation therapy because it places multiple, simultaneous pressures on the virus.

The second antiviral agent for the treatment of HIV, in one embodiment, can be a protease inhibitor, an HIV-integrase inhibitor, a chemokine inhibitor, or a reverse transcriptase inhibitor (“RTI”), the latter of which can either be a synthetic nucleoside reverse transcriptase inhibitor (“NRTI”) or a non-nucleoside reverse transcriptase inhibitor (“NNRTI”). In other embodiments, a second or third compound may be a pyrophosphate analog or a fusion-binding inhibitor. A list compiling resistance data collected in vitro and in vivo for certain antiviral compounds is found in Schinazi et al., Mutations in retroviral genes associated with drug resistance, International Antiviral News, 1997, 5(8).

In certain embodiments, the oxo-pyrimidine compound is administered in combination and/or alternation with FTC (2′,3′-dideoxy-3′-thia-5-fluorocytidine); 141W94 (amprenavir, Glaxo Wellcome, Inc.); Viramune (nevirapine); Rescriptor (delavirdine); DMP-266 (efavirenz); DDI (2′,3′-dideoxyinosine); 3TC (3′-thia-2′,3′-dideoxycytidine); DDC (2′,3′-dideoxycytidine), abacavir (1592U89), which is (1S,4R)-4-[(2-amino-6-cyclopropyl-amino)-9H-purin-9-yl]-2-cyclopentene-1-methanol succinate, D4T, or AZT.

Other examples of antiviral agents that can be used in combination and/or alternation with the compounds disclosed herein include, but are not limited to, foscarnet; carbovir; acyclovir; interferon, and β-D-dioxolane nucleosides such as β-D-dioxolanylguanine (DXG), β-D-dioxolanyl-2,6-diaminopurine (DAPD), and β-D-dioxolanyl-6-chloropurine (ACP).

Examples protease inhibitors that can be used in combination and/or alternation with the compounds disclosed herein include, but are not limited to indinavir ({1(1S,2R),5(S)}-2,3,5-trideoxy-N-(2,3-dihydro-2-hydroxy-1H-inden-1-yl)-5-[2-[[(1,1-dimethylethyl)amino]carbonyl]-4-(3-pyridinylmethyl)-1-piperazinyl]-2-(phenylmethyl)-D-erythro-pentoamide sulfate; Merck & Co., Inc.); nelfinavir (Agouron); ritonavir (Abbott Labs), saquinavir (Roche); and DMP-450 {[4R-4(r-a,5-a,6-b,7-6)-hexahydro-5,6-bis(hydroxy)-1,3-bis(3-amino)-phenyl]methyl-4,7-bis(phenylmethyl)-2H-1,3-diazepin-2-one}-bismesylate (Triangle Pharmaceuticals, Inc.).

The following drugs have been approved by the FDA or are currently or have been in clinical trials for use in the treatment of HIV infection, and therefore in one embodiment, can be used in combination and/or alternation with the compounds of the present invention.

| Drug Name | Manufacturer |
|---|---|
| 3TC, Epivir ® brand lamivudine | GlaxoSmithKline |
| abacavir generic Ziagen ®, ABC, or 1592U89 | GlaxoSmithKline |
| ABC, Ziagen ® brand abacavir, or 1592U89 | GlaxoSmithKline |
| ABT-378/r, or Kaletra ® brand lopinavir/ritonavir | Abbott Laboratories |
| AG-1549, S-1153, or capravirine (CPV) | Pfizer |
| AG1661, Remune ® brand HIV-1 Immunogen, or Salk vaccine | Immune Response Corp. |
| Agenerase ® brand amprenavir (APV), 141W94, or VX-478 | GlaxoSmithKline |
| aldesleukin generic Proleukin ®, or Interleukin-2 (IL-2) | Chiron Corporation |
| amdoxovir, or DAPD | Gilead Sciences |
| amprenavir generic Agenerase ®, APV, 141W94, or VX-478 | GlaxoSmithKline |
| APV, Agenerase ® brand amprenavir, 141W94, or VX-478 | GlaxoSmithKline |
| atazanavir generic Reyataz ™, or BMS-232632 | Bristol-Myers Squibb |
| AZT, Retrovir ® brand zidovudine (ZDV) | GlaxoSmithKline |
| Bis(POC) PMPA, Viread ® brand tenofovir DF | Gilead Sciences |
| BMS-232632, or Reyataz ™ brand atazanavir | Bristol-Myers Squibb |
| BMS-56190, or DPC-083 | Bristol-Myers Squibb |
| calanolide A | Sarawak Medichem |
| capravirine (CPV), AG-1549, or S-1153 | Pfizer |
| Combivir ® brand zidovudine + lamivudine, or AZT + 3TC | GlaxoSmithKline |
| CPV (capravirine), AG-1549, or S-1153 | Pfizer |
| Crixivan ® brand indinavir (IDV), or MK-639 | Merck & Co. |
| d4T, Zerit ® brand stavudine, or BMY-27857 | Bristol-Myers Squibb |
| DAPD, or amdoxovir | Gilead Sciences |
| ddC, or Hivid ® brand zalcitabine | Hoffmann-La Roche |
| ddI, Videx ® brand didanosine, or BMY-40900 | Bristol-Myers Squibb |
| delavirdine generic Rescriptor ®, DLV, or U-90152S/T | Pfizer |
| didanosine generic Videx ®, ddI, or BMY-40900 | Bristol-Myers Squibb |
| DLV, Rescriptor ® brand delavirdine, or U-90152S/T | Pfizer |
| DPC-083, or BMS-56190 | Bristol-Myers Squibb |
| Droxia ® brand hydroxyurea (HU) | Bristol-Myers Squibb |
| efavirenz generic Sustiva ®, or EFV | Bristol-Myers Squibb |
| EFV, Sustiva ® brand efavirenz | Bristol-Myers Squibb |
| emtricitabine generic Emtriva ™, or FTC | Gilead Sciences |
| Emtriva ® brand emtricitabine, or FTC | Gilead Sciences |
| enfuvirtide generic Fuzeon ™, or T-20 | Trimeris and Hoffmann-La |
| | Roche |
| Epivir ® brand lamivudine, or 3TC | GlaxoSmithKline |
| epoetin alfa (erythropoietin) generic Procrit ® | Ortho Biotech |
| erythropoietin (epoetin alfa) generic Procrit ® | Ortho Biotech |
| Fortovase ® brand saquinavir (Soft Gel Cap), or SQV (SGC) | Hoffmann-La Roche |
| fosamprenavir, or GW-433908, or VX-175 | GlaxoSmithKline |
| FTC, or Emtriva ® brand emtricitabine | Gilead Sciences |
| Fuzeon ™ brand enfuvirtide, or T-20 | Trimeris and Hoffmann-La |
| | Roche |
| GW-433908, or fosamprenavir, or VX-175 | GlaxoSmithKline |
| HE2000, or alpha-epibromide | HollisEden |
| | Pharmaceuticals |
| HIV-1 Immunogen generic Remune ®, Salk vaccine, or AG1661 | Immune Response Corp. |
| Hivid ® brand zalcitabine, or ddC | Hoffmann-La Roche |
| HU, or Droxia ® brand hydroxyurea | Bristol-Myers Squibb |
| hydroxyurea generic Droxia ®, or HU | Bristol-Myers Squibb |
| IDV, Crixivan ® brand indinavir, or MK-639 | Merck & Co. |
| IL-2 (Interleukin-2), or Proleukin ® brand aldesleukin | Chiron Corporation |
| indinavir generic Crixivan ®, IDV, or MK-639 | Merck & Co. |
| Interleukin-2 (IL-2), or Proleukin ® brand aldesleukin | Chiron Corporation |
| Invirase ® brand saquinavir (Hard Gel Cap), SQV (HGC), or | Hoffmann-La Roche |
| Ro-31-8959 | |
| Kaletra ® brand lopinavir/ritonavir, or ABT-378/r | Abbott Laboratories |
| lamivudine generic Epivir ®, or 3TC | GlaxoSmithKline |
| lopinavir/ritonavir generic Kaletra ®, or ABT-378/r | Abbott Laboratories |
| MK-639, Crixivan ® brand indinavir (IDV) | Merck & Co. |
| nelfinavir generic Viracept ®, NFV, or AG-1343 | Pfizer |
| nevirapine generic Viramune ®, NVP, or BI-RG-587 | Boehringer Ingelheim |
| NFV, Viracept ® brand nelfinavir, or AG-1343 | Pfizer |
| Norvir ® brand ritonavir (RTV), or ABT-538 | Abbott Laboratories |
| NVP, Viramune ® brand nevirapine, or BI-RG-587 | Boehringer Ingelheim |
| PNU-140690, or tipranavir | Boehringer Ingelheim |
| PRO-542 | Progenics |
| | Pharmaceuticals |
| Procrit ® brand epoetin alfa (erythropoietin) | Ortho Biotech |
| Proleukin ® brand aldesleukin, or Interleukin-2 (IL-2) | Chiron Corporation |
| Remune ® brand HIV-1 Immunogen, or Salk vaccine | Immune Response Corp. |
| Rescriptor ® brand delavirdine (DLV), or U-90152S/T | Pfizer |
| Retrovir ® brand zidovudine (ZDV), or AZT | GlaxoSmithKline |
| Reyataz ™ brand atazanavir, or BMS-232632 | Bristol-Myers Squibb |
| ritonavir generic Norvir ®, RTV, or ABT-538 | Abbott Laboratories |
| RTV, Norvir ® brand ritonavir, or ABT-538 | Abbott Laboratories |
| Salk vaccine, Remune ® brand HIV-1 Immunogen, or AG1661 | Immune Response Corp. |
| saquinavir (Hard Gel Cap) generic Invirase ®, SQV (HGC), or | Hoffmann-La Roche |
| Ro-31-8959 | |
| saquinavir (Soft Gel Cap) generic Fortovase ®, or SQV (SGC) | Hoffmann-La Roche |
| SCH-C | Schering-Plough |
| Serostim ® brand somatropin | Serono Laboratories |
| somatropin generic Serostim ® | Serono Laboratories |
| SQV (HGC), Invirase ® brand saquinavir (Hard Gel Cap), or | Hoffmann-La Roche |
| Ro-31-8959 | |
| SQV (SGC), or Fortovase ® brand saquinavir (Soft Gel Cap) | Hoffmann-La Roche |
| stavudine generic Zerit ®, d4T, or BMY-27857 | Bristol-Myers Squibb |
| Sustiva ® brand efavirenz (EFV) | Bristol-Myers Squibb |
| T-1249 | Trimeris and |
| | Hoffmann-La Roche |
| T-20, or Fuzeon ™ brand enfuvirtide | Trimeris and |
| | Hoffmann-La Roche |
| TDF, tenofovir DF generic Viread ™, or Bis(POC) PMPA | Gilead Sciences |
| tenofovir DF (TDF) generic Viread ®, Bis(POC) PMPA | Gilead Sciences |
| tipranavir, or PNU-140690 | Boehringer Ingelheim |
| TMC-114 | Tibotec-Virco Group |
| TMC-125 | Tibotec-Virco Group |
| Trizivir ® brand abacavir + zidovudine + lamivudine (ABC + AZT + 3TC) | GlaxoSmithKline |
| Videx ® brand didanosine, ddI, or BMY-40900 | Bristol-Myers Squibb |
| Videx ® EC brand didanosine (ddI): delayed-release capsules | Bristol-Myers Squibb |
| Viracept ® brand nelfinavir (NFV), or AG-1343 | Pfizer |
| Viramune ® brand nevirapine (NVP), or BI-RG-587 | Boehringer Ingelheim |
| Viread ® brand tenofovir DF, or Bis(POC) PMPA | Gilead Sciences |
| VX-175, or fosamprenavir, or GW-433908 | GlaxoSmithKline |
| zalcitabine generic Hivid ®, or ddC | Hoffmann-La Roche |
| ZDV, Retrovir ® brand zidovudine, or AZT | GlaxoSmithKline |
| Zerit ® brand stavudine, d4T, or BMY-27857 | Bristol-Myers Squibb |
| Ziagen ® brand abacavir (ABC), or 1592U89 | GlaxoSmithKline |
| zidovudine generic Retrovir ®, AZT, or ZDV | GlaxoSmithKline |

The following drugs have been approved by the FDA for use in the treatment of complications of HIV infection and AIDS, which can be used in combination and/or alternation with the compounds of the present invention.

| | | | Manufacturer |
|---|---|---|---|
| Brand Name | Generic Name | Use | Name |
| Abelcet, | Amphotericin B, ABLC | antifungal for aspergillosis | various |
| Ambisome | | | |
| Bactrim, Septra | sulfamethoxazole and | antiprotozoal antibiotic for | various |
| | trimethoprim | Pneumocystis carinii | |
| | | pneumonia treatment and | |
| | | prevention | |
| Biaxin, Klacid | clarithromycin | antibiotic for | Abbott |
| | | Mycobacterium avium | Laboratories |
| | | prevention and treatment | |
| Cytovene | ganciclovir, DHPG | antiviral for CMV retinitis | Roche |
| DaunoXome | daunorubicin-liposomal | chemotherapy for Kaposi's | Gilead |
| | | sarcoma | |
| Diflucan | fluconazole | antifungal for candidiasis, | Pfizer |
| | | cryptococcal meningitis | |
| Doxil | doxorubicin | chemotherapy for Kaposi's | Ortho Biotech |
| | hydrochloride-liposomal | sarcoma | |
| Famvir | famciclovir | antiviral for herpes | Novartis |
| Foscarnet | foscavir | antiviral for herpes, CMV | Astra |
| | | retinitis | Pharmaceuticals |
| Gamimune N | immune globulin, | immune booster to | Bayer |
| | gamma globulin, IGIV | prevent bacterial | Biologicals |
| | | infections in children | |
| Intron A | interferon alfa-2b | Karposi's sarcoma, | Schering |
| | | hepatitis C | |
| Marinol | dronabinol | treat appetite loss | Roxane |
| | | | Laboratories |
| Megace | megestrol acetate | treat appetite, weight loss | Bristol Myers- |
| | | | Squibb |
| Mepron | atovaquone | antiprotozoal antibiotic for | GlaxoSmithKline |
| | | Pneumocystis carinii | |
| | | pneumonia treatment and | |
| | | prevention | |
| Mycobutin, | rifabutin | antimycobacterial | Adria |
| Ansamycin | | antibiotic for | Pharmaceuticals |
| | | Mycobacterium avium | |
| | | prevention | |
| NebuPent | pentamidine | antiprotozoal antibiotic for | Fujisawa |
| | | Pneumocystis carinii | |
| | | pneumonia prevention | |
| Neutrexin | trimetrexate glucuronate | antiprotozoal antibiotic for | MedImmune |
| | and leucovorin | Pneumocystis carinii | |
| | | pneumonia treatment | |
| Panretin gel | alitretinoin gel 0.1% | AIDS-related Karposi's | Ligand |
| | | sarcoma | Pharmaceuticals |
| Procrit, Epogen | erythropoetin, EPO | treat anemia related to | Amgen |
| | | AZT therapy | |
| Roferon A | interferon alfa-2a | Karposi's sarcoma and | Roche |
| | | hepatitis C | |
| Serostim | somatropin rDNA | treat weight loss | Serono |
| Sporanox | itraconazole | antifungal for | Janssen |
| | | blastomycosis, | Pharmaceuticals |
| | | histoplasmosis, | |
| | | aspergillosis, and | |
| | | candidiasis | |
| Taxol | paclitaxel | Karposi's sarcoma | Bristol Myers- |
| | | | Squibb |
| Valcyte | valganciclovir | antiviral for CMV retinitis | Roche |
| Vistide | cidofovir, HPMPC | antiviral for CMV retinitis | Gilead |
| Vitrasert | ganciclovir insert | antiviral for CMV retinitis | Bausch & Lomb |
| implant | | | |
| Vitravene | fomivirsen sodium | antiviral for CMV retinitis | Isis |
| intravitreal | injection | | Pharmaceuticals |
| injectable | | | |
| Zithromax | azithromycin | antibiotic for | Pfizer |
| | | Mycobacterium avium | |

Several products have been allowed to proceed as Investigational New Drugs (IND) by the FDA for the treatment of complications of FHV infection and AIDS. Therefore, the following drugs can be used in combination and/or alternation with the compounds of the present invention.
Trimetrexate glucuronate for the treatment of Pneumocystis carinii pneumonia in AIDS patients who cannot tolerate standard forms of treatment.
Ganciclovir for the treatment of cytomegalovirus retinitis in AIDS patients.
Aerosolized pentamidine for the prevention of Pneumocystis carinii pneumonia in AIDS patients.
Erythropoietin for the treatment of zidovudine-related anemia.
Atovaquone for the treatment of AIDS patients with Pneumocystis carinii pneumonia who are intolerant or unresponsive to trimethoprim-sulfamethoxazole.
Rifabutin for prophylaxis against Mycobacterium avium complex bacteremia in AIDS patients.
Vistide intravenous cidofovir for HIV-infected persons with relapsing cytomegalovirus (CMV) retinitis that has progressed despite treatment (Hoffmann-La Roche).
Serostim, a mammalian derived recombinat human growth hormone, for the treatment of AIDS-related wasting (Serono Laboratories).

In general, during alternation therapy, an effective dosage of each agent is administered serially. During combination therapy, effective dosages of two or more agents are administered together. Dosages administered depend upon factors such as absorption, biodistribution, metabolism and excretion rates for each drug as well as other factors known to those skilled in the art. It is to be noted that dosage amounts will vary with the severity of the condition to be alleviated, the age, weight, and general physical condition of the subject who receives the drug. It is to be understood further that for any particular subject, specific dosage regimens and schedules should be adjusted over time according to the response of the subject to the drug, the needs of the subject, and the professional judgment of the person administering or supervising the administration of the compositions. Examples of suitable dosage ranges for anti-HIV compounds, including nucleoside derivatives such as, for example, D4T, DDI and 3TC, or protease inhibitors like nelfinavir and indinavir, are to be found in the scientific literature and Physicians' Desk Reference. Suggested ranges for effective dosages of the compounds of the present invention are guidelines only, and are not intended to limit the scope or use of the invention.

The disclosed combination and alternation regimen are useful in the treatment and prevention of retroviral infections and other related conditions, such as, for example, AIDS-related complex (ARC), persistent generalized lymphadenopathy (PGL), AIDS-related neurological conditions, anti-HIV antibody position and HIV-positive conditions, Kaposi's sarcoma, thrombocytopenia purpurea, and opportunistic infections. In addition, these compounds or formulations can be used prophylactically to prevent or retard the progression of clinical illness in individuals who are anti-HIV antibody or HIV-antigen positive, or who have been exposed to HIV.

Pharmaceutical Compositions

The oxo-pyrimidine compounds of the present invention can be administered to a subject in need thereof, optionally in combination or alternation with another anti-HIV or anti-retroviral agent, and/or with a pharmaceutically acceptable carrier, diluent or excipient. In one embodiment, a subject infected with HIV may be treated by administering to that subject an effective amount of a oxo-pyrimidine derivative, a salt, prodrug, stereoisomer or tautomer thereof, in the presence of a pharmaceutically acceptable carrier or diluent. For subjects with multiple drug resistance, the oxo-pyrimidine compound is administered either alone or in combination with one or more other anti-retroviral agents or anti-HIV agents. The active compounds may be administered by any appropriate route, for example, orally, parenterally, enterally, intravenously, intradermally, subcutaneously, percutaneously, transdermally, intranasally, topically or by inhalation therapy, and may be in solid, liquid or vapor form.

The active compound(s) are included within the pharmaceutically acceptable carrier, diluent or excipient in an amount sufficient to deliver to a patient a therapeutically effective amount of the active compound in order to inhibit viral replication in vivo, especially FHV replication, without causing serious toxic effects in a treated subject. By an “inhibitory amount” is meant an amount of active ingredient sufficient to halt viral replication as measured by, for example, an assay such as the ones referred to herein.

A preferred dose of the oxo-pyrimidine compound for all the conditions mentioned is in the range of from about 0.1 to 100 mg/kg of body weight per day, preferably from about 1 to 75 mg/kg of body weight per day, and even more preferably from about 1 to 20 mg/kg of body weight per day. The effective dosage range of the pharmaceutically acceptable derivatives is calculated based on the weight of the parent oxo-pyrimidine derivative to be delivered. If the derivative itself exhibits activity, then the effective dosage can be estimated as above using the weight of the derivative, or by other means known to those of skill in the art.

The compounds are conveniently administered in units of any suitable dosage form, including but not limited to one containing from about 7 to 3000 mg, preferably from about 70 to 1400 mg, and even more preferably from about 25 to 1000 mg of active ingredient per unit dosage form. For example, an oral dosage of from about 50 to 1000 mg is usually convenient.

Ideally, the active ingredient is administered to achieve peak plasma concentrations of the active compound of from about 0.02 to 70 μM, and preferably of from about 0.5 to 10 μM. For example, this can be achieved by intravenous injection of a 0.1 to 25% solution of active ingredient, optionally in saline, or administered as a bolus of active ingredient. It is to be understood that for any particular subject, specific dosage regimens should be adjusted over time to meet individual needs. The concentrations set forth here are exemplary only and are not intended to limit the scope or practice of the claimed composition. The active ingredient may be administered all at once, or may be divided into a number of smaller doses to be administered at varying intervals of time.

A preferred mode of administration of the active compound is oral. Oral compositions usually include an inert diluent or an edible carrier. They may be enclosed in gelatin capsules or compressed into tablets. For oral therapeutic administration, the active compound may be incorporated with excipients or formulated as solid dispersions or solid solutions, and used in the form of tablets, troches, or capsules. By a “solid dispersion” is meant a solid state comprising at least two components where one component is dispersed more or less evenly throughout the other component. By “solid solution” is meant a solid state comprising at least two components that are chemically and physically integrated to produce a homogeneous product. A solid solution is preferred over a solid dispersion because it more easily forms a liquid solution upon contact with an appropriate liquid medium, thereby increasing the bioavailability of a drug. Pharmaceutically compatible binding agents and/or adjuvant materials also may be included as part of this composition.

The tablets, pills, capsules, troches and the like may contain any of the following ingredients or compounds of a similar nature: a binder such as microcrystalline cellulose, gum tragacanth or gelatin; an excipient such as starch or lactose; a disintegrating agent such as alginic acid, Primogel, or cornstarch; a lubricant such as magnesium stearate or Sterotes; a glidant such as colloidal silicon dioxide; a sweetening agent like sucrose of saccharin; and a flavoring agent, such as peppermint, methyl salicylate, or orange flavoring. When the dosage unit form is a capsule, it may contain a liquid carrier such as a fatty oil in addition to any material of the kinds given above. In addition, dosage unit forms may contain various other materials that modify the physical form of the dosage unit, such as, for example, coatings of sugar, shellac, or other enteric agents.

The oxo-pyrimidine compounds may be administered as a component of an elixir, suspension, syrup, wafer, chewing gum or the like. A syrup may contain sucrose as a sweetening agent, preservatives, dyes, colorings, and flavorings in addition to the active compounds.

The active compounds or their pharmaceutically acceptable salts or prodrugs can be mixed with other active materials that do not impair the desired action, or with materials that supplement the desired action, such as antibiotics, antifungals, anti-inflammatories, protease inhibitors, or other nucleoside or non-nucleoside antiviral agents. Solutions or suspensions used for parenteral, intradermal, subcutaneous, or topical application can include the following components: a sterile diluent such as water for injection, salilne solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates, or phosphates, and agents for the adjustment of tonicity such as sodium chloride or dextrose. A parenteral preparation normally will include sterile water and may be enclosed in ampoules, disposable syringes, or multiple dose vials made of glass or plastic.

If administered intravenously, preferred carriers are physiological saline, phosphate buffered saline (PBS), a glucose solution, or a mixed solution comprising glucose and saline. If administration is percutaneous, such as, for example, through the use of a patch or ointment, the associated carrier may comprise a penetration-enhancing agent and/or a suitable wetting agent which are not harmful to the skin. If inhalation or insufflation is the desired route of administration, then the composition of the present invention includes the compound in the form of a solution, suspension or dry powder that can be delivered through the oral and/or nasal orifices.

Liposomal suspensions, which include liposomes targeted to infected cells with monoclonal antibodies to viral antigens, also are preferred as pharmaceutically acceptable carriers. These may be prepared according to methods known to those skilled in the art, for example, as described in U.S. Pat. No. 4,522,811, which is incorporated herein by reference in its entirety. For example, liposomal formulations may be prepared by dissolving appropriate lipid(s) such as stearoyl phosphatidyl ethanolamine, stearoyl phosphatidyl choline, arachadoyl phosphatidyl choline, and cholesterol, in an inorganic solvent that later is evaporated, leaving behind a thin film of dried lipid on the surface of the container. An aqueous solution of the active compound, or a salt or prodrug thereof, is then introduced into the container. The container is swirled to free lipid material from its sides and to disperse lipid aggregates, thereby forming the liposomal suspension.

Processes for Preparing the Active Compounds

The oxo-pyrimidine compounds of the present invention can be synthesized by any means known to those skilled in the art. In particular, the processes disclosed in U.S. application Ser. No. 09/744,038 and U.S. application Ser. No. 10/001,868, both to Idenix Pharmaceuticals, Inc., and both hereby incorporated by reference in their entireties, may be used to synthesize the compounds of the present invention. The following non-limiting species of oxo-pyrimidine compounds can be synthesized by the processes provided below. It is to be understood that other syntheses known to those of skill in the art also may be used to prepare the compounds of the present invention. Moreover, certain steps within a process may be performed in a different order than that shown with the same result. For example, in Schemes 1 and 2, product compound 10 may be prepared from compound 4 by reacting 4 first with HNR₉R₉/base, then with NaOMe, and finally with HCl. All such alternative schemes are intended for inclusion herein. 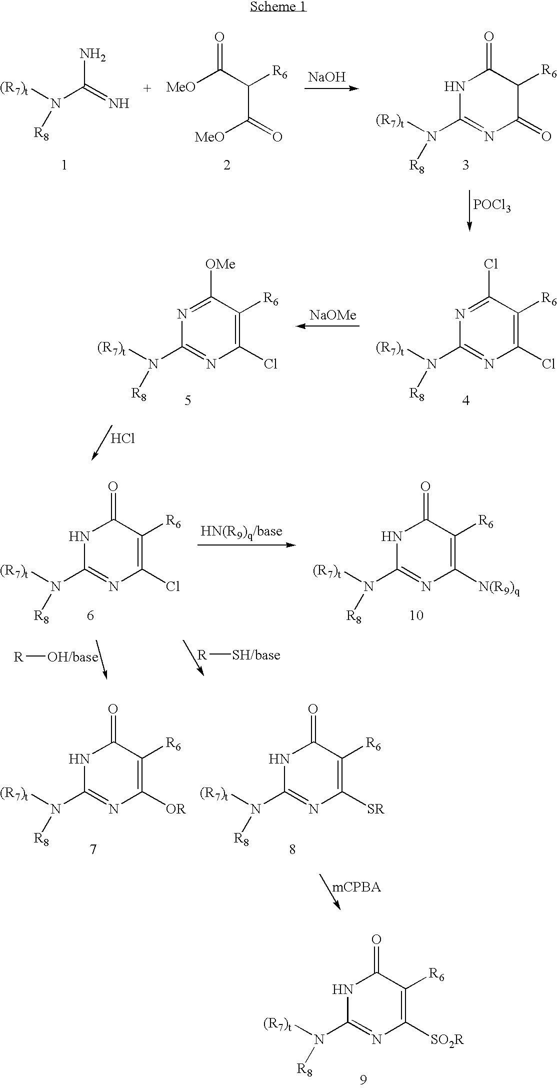
wherein R₆, (R₇)ₜ, R₈, and (R₉)_{q}, each independently, is as defined previously herein, and R is optionally substituted —(CH₂)ᵣ-phenyl. 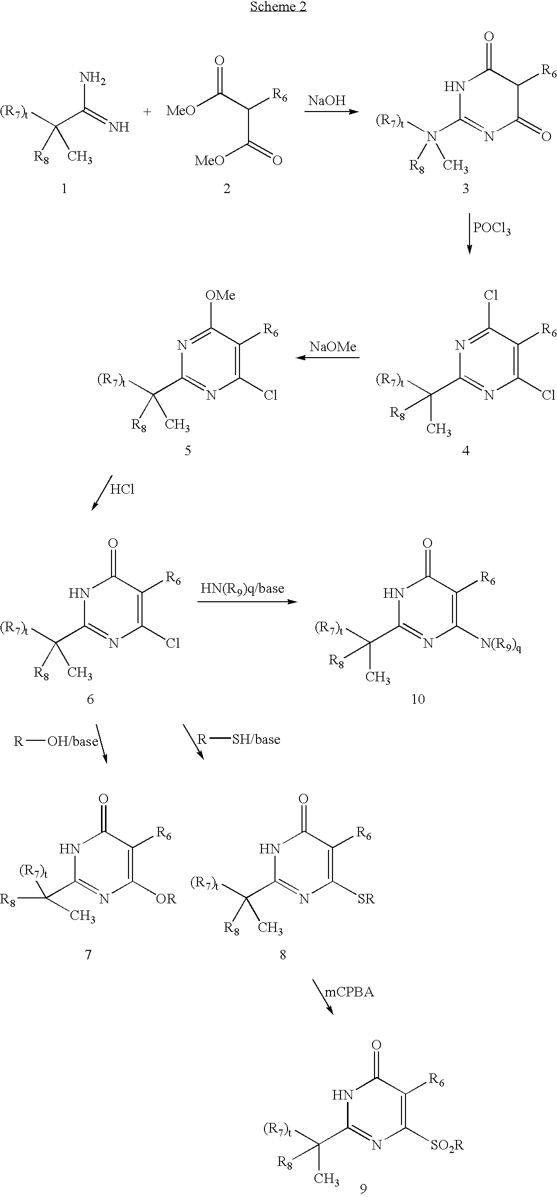
wherein R₆, (R₇)ₜ, R₈, and (R₉)_{q}, each independently, are as previously defined herein and R is optionally substituted —(CH₂)ᵣ-phenyl. 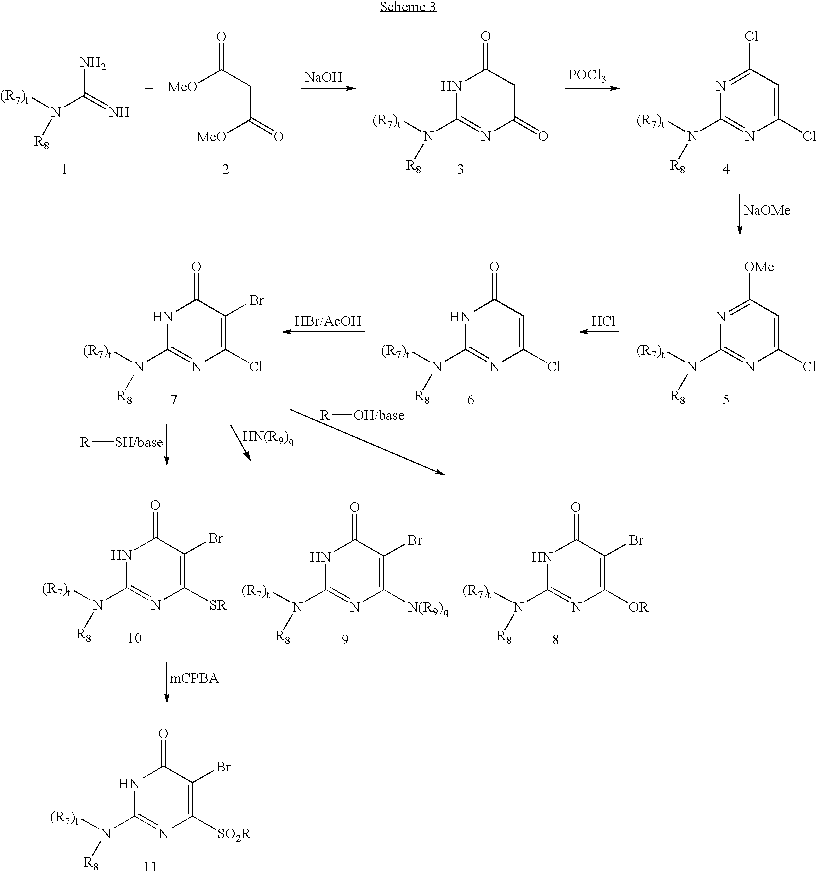
wherein each (R₇)ₜ, R₈, and (R₉)_{q}, independently, is as defined previously herein, and R is optionally substituted —(CH₂)ᵣ-phenyl. 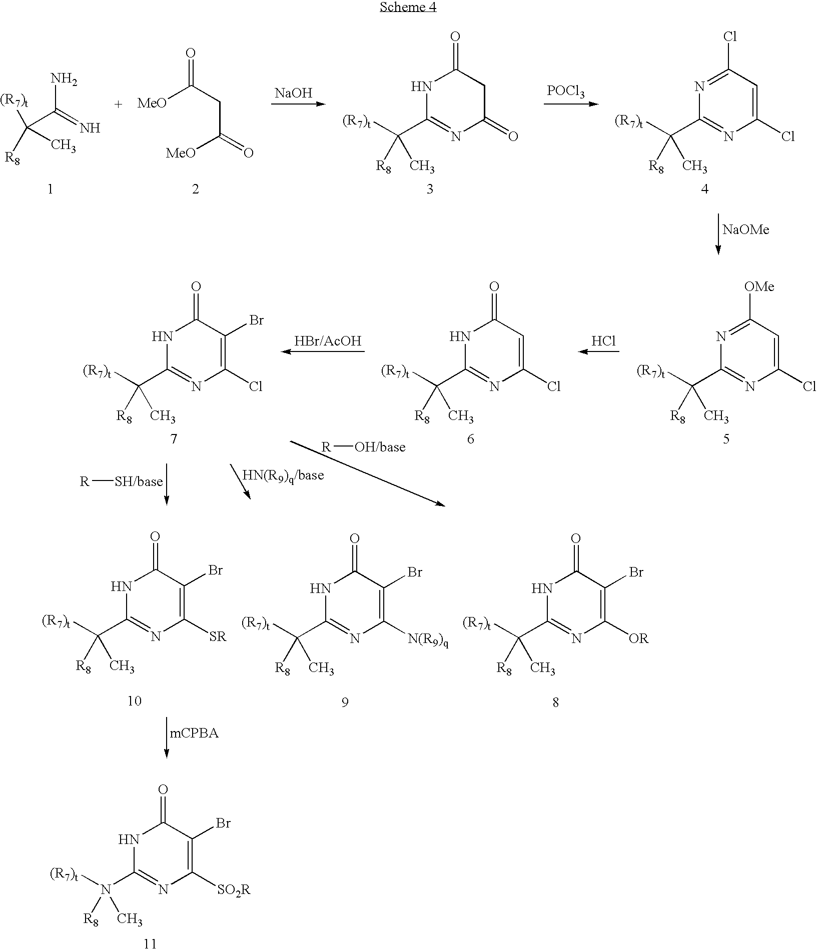
wherein (R₇)ₜ, R₈, and (R₉)_{q}, each independently, is as defined previously herein, and R is optionally substituted —(CH₂)ᵣ-phenyl. 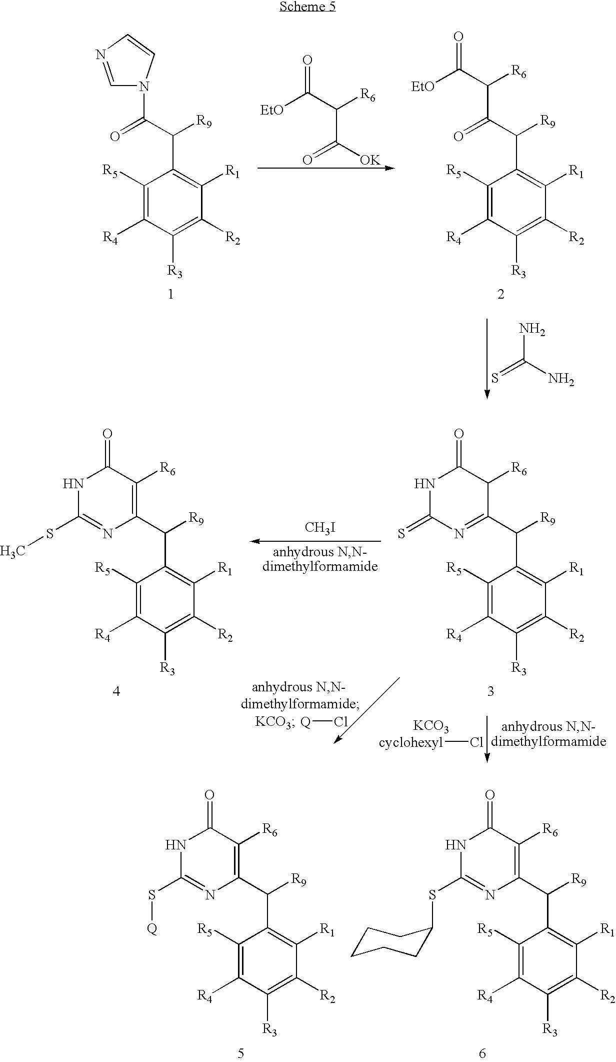
wherein each R₁, R₂, R₃, R₄, R₅, R₆, and R₉ independently, is as defined previously herein, and Q is —H; —C₁₋₆ alkyl, optionally having one or more heteroatoms including but not limited to O, N or S; —C₃₋₆ cycloalkyl, optionally having one or more heteroatoms including but not limited to O, N or S; or optionally substituted heterocycle, with the caveat that R may not be a 5- or 6-membered ring having one or more nitrogens as the only heteroatom. 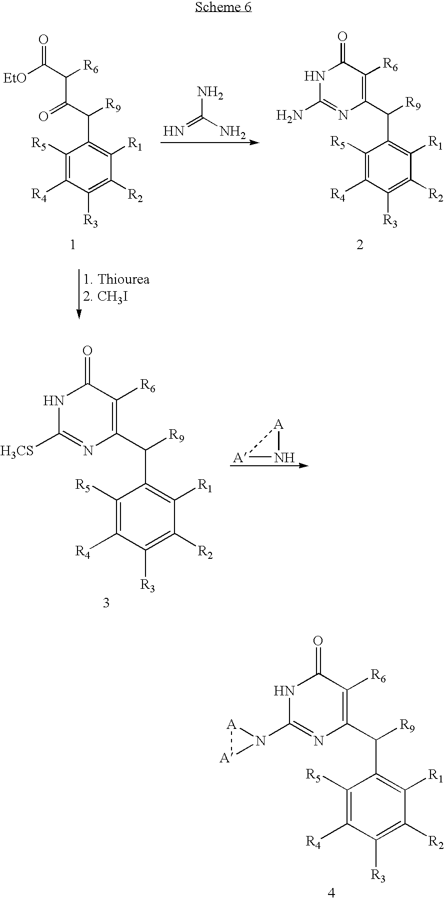
wherein each R₁, R₂, R₃, R₄, R₅, R₆ and R₉, independently, is as defined previously herein;
A is C₁₋₆ alkyl or amino;
— denotes a single or double bond; and
A′ is optionally substituted —C₁₋₆ alkyl, optionally having one or more heteroatoms including but not limited to O, N or S.
   Biological Activity Against Drug Resistant Strains of HIV

In one embodiment, the efficacy of an anti-HIV compound is measured in vitro by a rapid, sensitive, and automated assay that involves the reduction of 3-(4,5-dimethylthiazol-2-yl)-2,5-diphenyltetrazolium bromide (MTT). An HIV-transformed cell line that is highly permissive and selective for HIV infection, such as, for example, the T-4 cell line, MT-4, is chosen as the target cell line (Koyanagi et al., Int. J. Cancer, 1985, 36:445-451). In situ reduction of 3-(4,5-dimethylthiazol-2-yl)-2,5-diphenyl-tetrazolium bromide (MTT) as assessed spectrophotometrically is the standard by which the viability of both mock-infected cells and HIV-infected cells is measured. Inhibition of the HIV-induced cytopathic effect serves as the end-point. A 50% cytotoxic concentration (CC₅₀ in μM) is defined as the concentration of compound that reduces the absorbance of the mock-infected control sample by 50%. The percent efficacy of an anti-HIV compound is calculated by the formula (expressed as a %):
(OD_{HIV test compound})−(OD_{control})/(OD_{mock infected cells})−(OD_{control})
Here, (OD_{HIV test compound}) is the optical density measured for a specific amount of a test compound in HIV-infected cells; (OD_{control}) is the optical density measured for untreated HIV-infected, control cells; and (OD_{mock infected cells}) is the optical density measured for control, mock-infected cells that are untreated. Optical density values typically are assessed at 540 nm. The dosage of an anti-HIV test compound that provides 50% protection according to the preceding formula is defined as the 50% inhibitory concentration (IC₅₀ in μM). The selectivity index (SI) is defined as the ratio of the CC₅₀ to the IC₅₀.

In another embodiment, the p24 ELISA assay is used to determine the efficacy of an anti-HIV compound. This viral replication immunoassay measures the amount of p24 viral capsid (core) antigen present, and is available commercially from sources such as, for example, Coulter Corporation/Immunotech, Inc.® (Westbrook, Mich.).

Still other embodiments include a reverse trancriptase assay in which the amount of viral replication is measured by utilizing a homopolymer poly rA:oligo dT template primer system that quantifies the incorporation into cells of tritiated thymidine monophosphate by scintillation counting methods (Southern Research Institute, University of Alabama, Birmingham, Ala.); a syncytial inhibition assay that employs CEM-SS, HeLa-CD4, or HeLa-CD4-LTR-b-galactosidase cells having an immunofluorescent, chemiluminescent, or colorimetric endpoint; and an attachment-and fusion-inhibition assay that utilizes indicator cell lines and quantitation by chemiluminescent, calorimetric or microscopic evaluation (Southern Research Institute, University of Alabama, Birmingham, Ala.).

In one embodiment the oxo-pyrimidine compounds of the present invention do not exhibit significant cross resistance with other non-nucleoside reverse transcriptase inhibitors (NNRTIs), in that the compounds of the present invention display an EC₅₀ (in molar concentration) in a mutant HIV strain of less than approximately 50, 25, 10 or 1 μM concentration. In a preferred embodiment, the NNRTIs display an EC₅₀ in a mutant HIV strain of less than approximately 5, 2.5, 1 or 0.1 μM concentration. The degree of cross-resistance against a drug resistant strain of HIV is measured by assessing the EC₅₀ of the desired oxo-pyrimidine compound in the target mutated, i.e., drug resistant, virus.

Therefore, in another important embodiment of this invention, a method for treating a patient with a cross-resistant HIV is provided that includes administering an effective HIV-treatment amount of a oxo-pyrimidine compound, a salt, prodrug, stereoisomer or tautomer thereof.

The following examples are provided to illustrate the present invention, and are in no way intended to limit the scope of the invention.

### EXAMPLES

### Example 1

### Preparation of 5-alkyl-6-benzyl-3,4-dihydro-2-methylthiopyrimidin-4-one (Scheme 5)

The proper ethyl arylacetylacetate derivative (31.5 mmoles) was successively added to a stirred solution of sodium metal (0.063 g-atoms) in 50 mL of absolute ethanol (50 mL) and thiourea (43 mmoles). The mixture was heated while stirring at reflux for 5 hours. After cooling, the solvent was distilled in vacuo at 40-50° C. until dryness and the residue was dissolved in water (200 mL) and made acidic (pH 5) with 0.5 N acetic acid. The resulting precipitate, the crude 2-thiouracil derivative, was filtered under reduced pressure, washed with diethyl ether, vacuum dried at 80° C. for 12 h. and crystallized from an appropriate solvent to give (3).

To prepare compound 4, iodomethane (8 mmoles, 1.13 g) was added to a suspension containing the proper 2-thiouracil derivative (4 mmoles) in anhydrous N,N-dimethylformamide (2 mL), and the resulting mixture was stirred at room temperature until the starting material disappeared at the TLC control (silica gel; n-hexane: ethyl acetate: methanol 12:3:1). Then the reaction content was poured onto cold water (100 mL) and extracted with ethyl acetate (3×50 mL). The organic layers were collected, washed with a sodium thiosulfate solution (100 mL), brine (3×50 mL), dried and evaporated to provide crude 5-alkyl-6-benzyl-3,4-dihydro-2-methylthiopyrimidin-4-one (4) as a solid purified by crystallization.

### Example 2

### 5-alkyl-6-benzyl-3,4-dihydro-2-R-substituted thiopyrimidin-4-one (Scheme 5)

The proper ethyl arylacetylacetate derivative (31.5 mmoles) was successively added to a stirred solution of sodium metal (0.063 g-atoms) in 50 m]L of absolute ethanol (50 mL) and thiourea (43 mmoles). The mixture was heated while stirring at reflux for 5 hours. After cooling, the solvent was distilled in vacuo at 40-50° C. until dryness and the residue was dissolved in water (200 mL) and made acidic (pH 5) with 0.5 N acetic acid. The resulting precipitate, the crude 2-thiouracil derivative, was filtered under reduced pressure, washed with diethyl ether, vacuum dried at 80° C. for 12 h. and crystallized from an appropriate solvent to give (3).

Next, potassium carbonate (4.2 mmoles) and the proper alkyl halide (4.4 mmoles) were added to as suspension containing 2-thiouracil derivative (4 mmoles) in anhydrous N,N-dimethylforrnamide(2 mL). The resulting mixture was stirred at room temperature until the starting material disappeared at the TLC control (silica gel; n-hexane: ethyl acetate: methanol 12:3:1). Then the reaction content was poured onto cold water (100 mL) and extracted with ethyl acetate (3×50 mL). The organic layers were collected, washed with a sodium thiosulfate solution (100 mL), brine (3×50 mL), dried and evaporated to provide 5-alkyl-6-benzyl-3,4-dihydro-2-R-substituted-thiopyrimidin4-one (5) as crude material which was purified by column chromatography on silica gel (eluent: n-hexane: ethyl acetate: methanol 12:3:1) followed by crystallization. Physical and chemical data of representative compounds are provided in the examples that follow.

### Example 3

### 5-alkyl-6-benzyl-3,4-dihydro-2-cyclohexyl-thiopyrimidin-4-one (Scheme 5)

The proper ethyl arylacetylacetate derivative (31.5 mmoles) was successively added to a stirred solution of sodium metal (0.063 g-atoms) in 50 mL of absolute ethanol (50 mL) and thiourea (43 mmoles). The mixture was heated while stirring at reflux for 5 hours. After cooling, the solvent was distilled in vacuo at 40-50° C. until dryness and the residue was dissolved in water (200 mL) and made acidic (pH 5) with 0.5 N acetic acid. The resulting precipitate, the crude 2-thiouracil derivative, was filtered under reduced pressure, washed with diethyl ether, vacuum dried at 80° C. for 12 h. and crystallized from an appropriate solvent to give (3).

Next, potassium carbonate (4.2 mmoles) and the proper alkyl halide (4.4 mmoles) were added to as suspension containing 2-thiouracil derivative (4 mmoles) in anhydrous N,N-dimethylformamide (2 mL). The resulting mixture was stirred at 80° C. until the starting material disappeared at the TLC control (silica gel; n-hexane: ethyl acetate: methanol 12:3:1). Then the reaction content was poured onto cold water (100 mL) and extracted with ethyl acetate (3×50 mL). The organic layers were collected, washed with a sodium thiosulfate solution (100 mL), brine (3×50 mL), dried and evaporated to provide 5-alkyl-6-benzyl-3,4-dihydro-2-cyclohexyl-thiopyrimidin-4-one (6) as crude material which was purified by column chromatography on silica gel (eluent: n-hexane: ethyl acetate: methanol 12:3:1) followed by crystallization. Physical and chemical data of representative compounds are provided in the examples that follow.

### Example 4

### Preparation of 2-Cyclopentylamino-6-(2,6-difluorophenylmethyl)-3,4-dihydropyrimidin-4-(3H)-one (Scheme 6)

Cyclopentylamine (10 mL) was heated while stirring with 6-(2,6-difluorophenylmethyl)-3,4-dihydro-2-methylthiopyrimidin-4-(3H)-one (0.30 g., 1.12 mmol; prepared as reported in Scheme 5 or 6) in a sealed tube at 160° C. for 10 hours. After cooling the mixture was diluted with water (200 mL) and extracted with ethyl acetate (3×50 mL). The organic layers were collected, washed with brine (3×50 mL), dried and evaporated to furnish crude product, which was purified by chromatography on silica gel column (eluent: ethyl acetate/chloroform, 1:1 ratio).

% Yield: 74; formula: C₁₆H₁₇F₂N₃O; and molecular weight: 305.33.

### Example 5

### Synthesis of 2-Cyclopentylamino-6-(2,6-difluorophenylmethyl)-3,4-dihydro-5-methylpyrimidin-4-(3H)-one (Scheme 6)

Cyclopentylamine (10 mL) was heated while stirring with 6-(2,6-difluorophenylmethyl)-3,4-dihydro-5-methyl-2-methylthiopyrimidin-4-(3H)-one (0.30 g., 1.12 mmol; prepared as reported in Scheme 5 or 6) in a sealed tube at 160° C. for 10 hours. After cooling the mixture was diluted with water (200 mL) and extracted with ethyl acetate (3×50 mL). The organic layers were collected, washed with brine (3×50 mL), dried and evaporated to furnish crude product, which was purified by chromatography on silica gel column (eluent: ethyl acetate/chloroform, 1:1 ratio).

% Yield: 60; mp (° C.): 115-117; recrystallization solvent: n-hexane/cyclohexane; formula: C₁₇H₁₉F₂N₃O; and molecular weight: 319.35.

### Example 6

### Synthesis of 2-Cyclopentylamino-6-{1-(2,6-difluorophenyl)ethyl}3,4-dihydropyrimidin-4-(3H)-one (Scheme 6)

Cyclopentylamine (10 mL) was heated while stirring with 6-{1-(2,6-difluorophenyl)ethyl}-3,4-dihydro-5-methyl-2-methylthiopyrimidin-4-(3H)-one (0.30 g., 1.12 mmol; prepared as reported in Example 9,Scheme 9) in a sealed tube at 160° C. for 10 hours. After cooling the mixture was diluted with water (200 mL) and extracted with ethyl acetate (3×50 mL). The organic layers were collected, washed with brine (3×50 mL), dried and evaporated to furnish crude product, which was purified by chromatography on silica gel column (eluent: ethyl acetate/chloroform, 1:1 ratio) (See Scheme 5 or 6).

% Yield: 48; formula: C₁₇H₁₉F₂N₃O; MW=319.35.

### Example 7

### Synthesis of 2-Cyclopentylamino-6-{1-(2,6-difluorophenyl)ethyl}-3,4-dihydro-5-methylpyrimidin-4-(3H)-one (Scheme 6)

Cyclopentylamine (10 mL) was heated while stirring with 6-{1-(2,6-difluorophenyl)ethyl}-3,4-dihydro-5-methyl-2-methylthiopyrimidin-4-(3H)-one (0.30 g., 1.12 mmol; prepared as reported in Example 9,Scheme 9) in a sealed tube at 160° C. for 10 hours. After cooling the mixture was diluted with water (200 mL) and extracted with ethyl acetate (3×50 mL). The organic layers were collected, washed with brine (3×50 mL), dried and evaporated to furnish crude product, which was purified by chromatography on silica gel column (eluent: ethyl acetate/chloroform, 1:1 ratio). (See Scheme 5 or 6).

% Yield: 38; formula and molecular weight: C18H₂₁F₂N₃O, MW 333.38.

This invention has been described with reference to its preferred embodiments. Variations and modifications of the invention will be obvious to those skilled in the art from the foregoing detailed description of the invention. It is intended that all of these variations and modifications be included within the scope of this invention.

1. A compound of Formula (I): 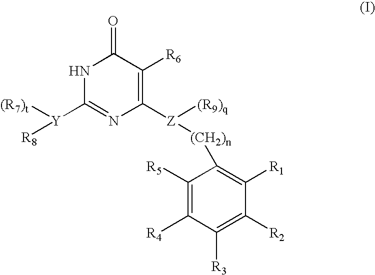
or a pharmaceutically acceptable salt, prodrug, N-oxide, quaternary amine, stereochemical isomer or tautomer thereof, wherein
a) each R₁, R₂, R₃, R₄, R₅, independently, is —H; —C₁₋₆ alkyl; —C_{2- 6}alkenyl; —C₂₋₆ alkynyl; optionally substituted aryl; -aralkyl; -halo; -haloalkyl; —CF₃; —NO₂; —CN; —CONH₂; —CONH—C₁₋₆ alkyl; —CON(C₁₋₆ alkyl)₂; —NH₂; —NH—C₁₋₆ alkyl; —N(C₁₋₆ alkyl)₂; —NHC(O)-alkyl; —NHSO₂—C₁₋₆ alkyl; —SO₂NH₂; —SO₂NH—C₁₋₆ alkyl; —SO₂N(C₁₋₆ alkyl)₂; —SO₂NH-aryl; and —OW or —SW, where W is —H, or optionally substituted alkyl, aryl or aralkyl;
b) R₇ and R₈, each independently, is —H; —C₁₋₆ straight or branched chain alkyl; —C₂₋₆ alkenyl; —C₂₋₆ alkynyl; —C₃₋₆ cycloalkyl; 3-7 membered heterocycle; -aralkyl; -aryl; -haloalkyl; —CN; —CONH₂; —CONH—C₁₋₆ alkyl; —CON(C₁₋₆ alkyl)₂; —NH₂; —NH—C₁₋₆ alkyl; —N(C₁₋₆ alkyl)₂; —NHC(O)-C₁₋₆ alkyl; —NHSO₂—C₁₋₆alkyl; —(CH₂),-phenyl; —SO₂NH₂; —SO₂NH—C₁₋₆ alkyl; —SO₂N(C₁₋₆ alkyl)₂; —SO₂NH-aryl; any of which may be substituted or unsubstituted; or
R₇ and R₈, each independently is —OW or —SW when Z is N or C, and where W is —H or optionally substituted alkyl, aryl or aralkyl; or
R₇ and R₈ together with the Y substituent to which they are attached, form an alicyclic, aryl or heterocyclic ring that optionally may have one or more O, S or N atoms, and further may be substituted or unsubstituted;
c) R₆ is —H; -halo; —C₁₋₆ straight or branched chain alkyl; —C₁₋₆ alkenyl; —C₁₋₆ alkynyl; —C₃₋₆cycloalkyl; 3-7 membered heterocycle; -aryl; -aralkyl; -halo; -haloalkyl; —CN; —CF₃; NO₂; —CONH₂; —CONH—C 1₆ alkyl; —CON(C₁₋₆ alkyl)₂; —NH₂; —NH—C₁₋₆ alkyl; —N(C₁₋₆ alkyl)₂; —NHC(O)-C₁₋₆ alkyl; —NHSO₂—C₁₋₆ alkyl; —SO₂NH₂; —SO₂NH—C₁₋₆ alkyl; —SO₂N(C₁₋₆ alkyl)₂; —SO₂NH-aryl; any of which may be substituted or unsubstituted; or
R₆ is —OW or —SW when Z is N or C, and where W is —H or optionally substituted alkyl, aryl or aralkyl;
d) R₉ is —H; —C₁₋₆straight or branched chain alkyl; —C₁₋₆ alkenyl; —C₁₋₆ alkynyl; —C₃₋₆ cycloalkyl; 3-7 membered heterocycle; -aryl; -aralkyl; -haloalkyl; —CN; —CONH₂; —CONH—C₁₋₆ alkyl; —CON(C₁₋₆ alkyl)₂; —NH₂; —NH—C₁₋₆ alkyl; —N(C₁₋₆ alkyl)₂; —NHC(O)—C₁₋₆ alkyl; —NHSO₂—C₁₋₆ alkyl; —SO₂NH₂; —SO₂NH—C₁₋₆ alkyl; —SO₂N(C₁₋₆ alkyl)₂; —SO₂NH-aryl; any of which may be substituted or unsubstituted; or
R₉ is —OW or —SW when Z is N or C, and where W is —H or optionally substituted alkyl, aryl or aralkyl; or
e) R₆ and R₉ taken together with the pyrimidinone ring and Z substituent to which they are attached respectively, form a bicyclic heterocycle that optionally may have additional O, S or N atoms, and further may be substituted or unsubstituted; and
where t is 1, 2, or 3, then each occurrence of R₇ is independently selected from the substituents listed herein for R₇;
where q is 1, 2 or 3, then each occurrence of R₉ is independently selected from the substituents listed herein for R₉;
f) Y is O, S, or SO₂, and t is 0; or
Y is CH or N, and t is 1; or
Y is C or N⁺ (with a suitable counter ion), and t is 2;
g) Z is O, S, or SO₂, and q is 0; or
Z is CH orN, and q is 1; or
Z is C or N⁺ (with a suitable counter ion), and q is 2; and
h) n is 0-4.

2. The compound of claim 1 wherein R₆ is Br or CH₃.

3. The compound of claim 1 wherein Z is O.

4. The compound of claim 1 wherein Z is C or CH.

5. The compound of claim 1 wherein Y is N.

6. A compound of Formula (Ia): 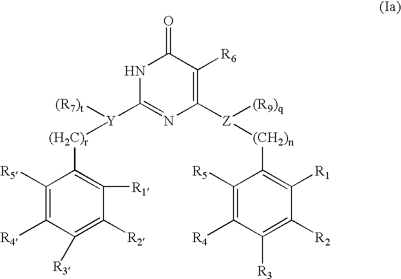
or a pharmaceutically acceptable salt, prodrug, N-oxide, quaternary amine, stereochemical isomer or tautomer thereof, wherein
a) each R₁, R₂, R₃, R₄R₅, R_{1′}, R_{2′}, R_{3′}, R_{4′} and R₅′ independently, is —H; —C₁₋₆ alkyl; —C₂₋₆ alkenyl; —C₂₋₆ alkynyl; -aralkyl; optionally substituted aryl; -halo; -haloalkyl; —NO₂; —CN; —CONH₂; —CONH—C₁₋₆ alkyl; —CON(C₁₋₆ alkyl)₂; —NH₂; ; —NH—C₁₋₆ alkyl; —N(C₁₋₆ alkyl)₂; —NHC(O)-alkyl; —NHSO₂—C₁₋₆ alkyl; —SO₂NH₂; —SO₂NH—C₁₋₆ alkyl; —SO₂N(C₁₋₆ alkyl)₂; —SO₂NH-aryl; and —OW or —SW, where W is —H, or optionally substituted alkyl, aryl or aralkyl;
b) R₆ and R₇, each independently, is H; C₁₋₆ straight or branched chain alkyl; —C₂₋₆ alkenyl; —C₂₋₆ alkynyl; aralkyl; halo; haloalkyl; C₃₋₆ cycloalkyl; 3-7 membered heterocycle; or -aryl; —NO₂; —CONH₂; —CONH—C₁₋₆ alkyl; —CN; —CON(C₁₋₆ alkyl)₂; —NH₂; —NH—C₁₋₆ alkyl; —N(C₁₋₆ alkyl)₂; —NHC(O)—C₁₋₆ alkyl; —NHSO₂—C₁₋₆ alkyl; —SO₂NH₂; —SO₂NH—C₁₋₆ alkyl; —SO₂N(C₁₋₆ alkyl)₂; —SO₂NH-aryl; —(CH₂)ₙ-phenyl; any of which may be substituted or unsubstituted; or
R₆ and R₇, each independently, is —OW or —SW when Z is N or preferably C, and where W is —H or optionally substituted alkyl, aryl or aralkyl;
c) R₉ is —H; —C₁₋₆ straight or branched chain alkyl; —C₁₋₆ alkenyl; —C₁₋₆ alkynyl; —C₃₋₆ cycloalkyl; 3-7 membered heterocycle; -aryl; -aralkyl; -haloalkyl; —CN; —CONH₂; —CONH—C₁₋₆alkyl; —CON(C₁₋₆ alkyl)₂; —NH₂; —NH—C₁₋₆ alkyl; —N(C₁₋₆ alkyl)₂; —NHC(O)—C₁₋₆ alkyl; —NHSO₂—C₁₋₆ alkyl; —SO₂NH₂; —SO₂NH—C₁₋₆ alkyl; —SO₂N(C₁₋₆ alkyl)₂; —SO₂NH-aryl; any of which may be substituted or unsubstituted; or
R₉ is —OW or —SW when Z is N or preferably C, and where W is —H or optionally substituted alkyl, aryl or aralkyl; or
d) R₆ and R₉ taken together with the pyrimidinone ring and Z substituent to which they are attached, form a bicyclic heterocycle that optionally may have additional O, S or N atoms, and which further may be substituted or unsubstituted; and
where t is 1, 2, or 3, then each occurrence of R₇ is independently selected from the substituents listed herein for R₇;
where q is 1, 2 or 3, then each occurrence of R₉ is independently selected from the substituents listed herein or R₉;
e) r is 0-4;
f) Y is O, S, or SO₂, and t is 0; or
Y is CH or N, and t is 1; or
Y is C or N⁺ (with a suitable counter ion), and t is 2;
g) Z is O, S, or SO₂, and q is 0; or
Z is CH or N, and q is 1; or
Z is C or N⁺ (with a suitable counter ion), and q is 2; and
h) n is 0-4.

7. The compound of claim 6 wherein R₆ is Br or CH₃.

8. The compound of claim 6 wherein Z is C.

9. The compound of claim 6 wherein Z is O.

10. The compound of claim 6 wherein Y is N.

11. A compound of Formula (a): 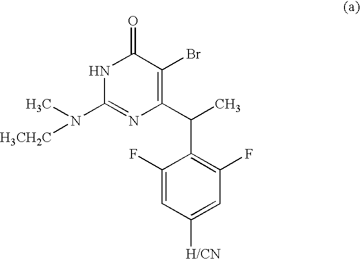
or a pharmaceutically acceptable salt, prodrug, stereoisomer, tautomer, N-oxide or quaternary amine thereof.

12. A compound of Formula (b): 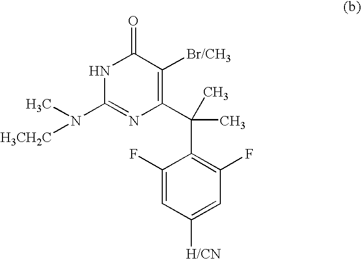
or a pharmaceutically acceptable salt, prodrug, stereoisomer, tautomer, N-oxide or quaternary amine thereof.

13. A compound of Formula (c): 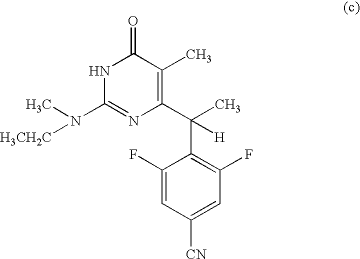
or a pharmaceutically acceptable salt, prodrug, stereoisomer, tautomer, N-oxide or quaternary amine thereof.

14. A compound of Formula (d): 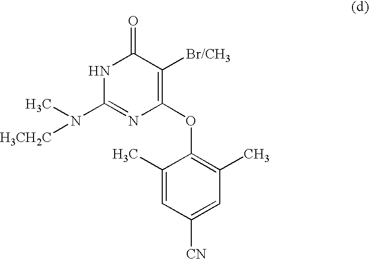
or a pharmaceutically acceptable salt, prodrug, stereoisomer, tautomer, N-oxide or quaternary amine thereof.

15. A compound of Formula (e): 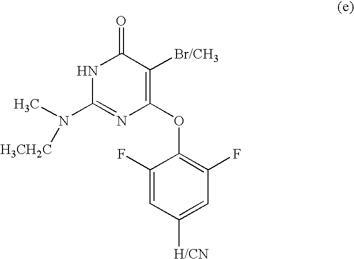
or a pharmaceutically acceptable salt, prodrug, stereoisomer, tautomer, N-oxide or quaternary amine thereof.

16. A compound of Formula (f): 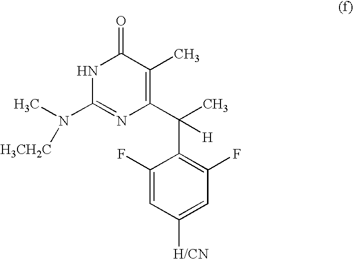
or a pharmaceutically acceptable salt, prodrug, stereoisomer, tautomer, N-oxide or quaternary amine thereof.

17. A compound of Formula (g): 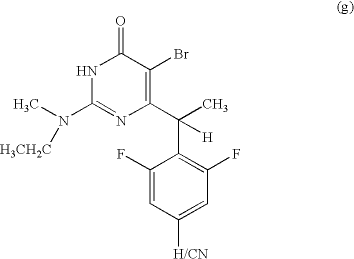
or a pharmaceutically acceptable salt, prodrug, stereoisomer, tautomer, N-oxide or quaternary amine thereof.

18. A compound of Formula (h): 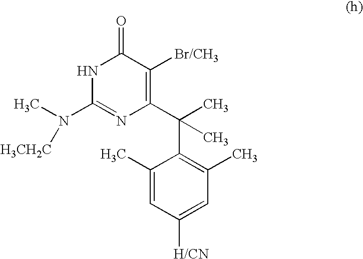
or a pharmaceutically acceptable salt, prodrug, stereoisomer, tautomer, N-oxide or quaternary amine thereof.

19. A compound of Formula (i): 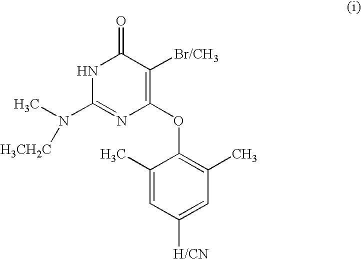
or a pharmaceutically acceptable salt, prodrug, stereoisomer, tautomer, N-oxide or quaternary amine thereof.

20. A compound of Formula (j): 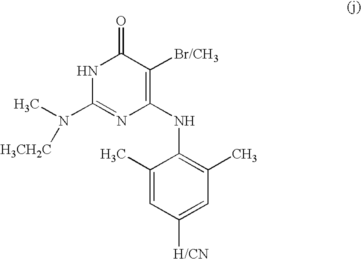
or a pharmaceutically acceptable salt, prodrug, stereoisomer, tautomer, N-oxide or quaternary amine thereof.

21. A compound of Formula (k): 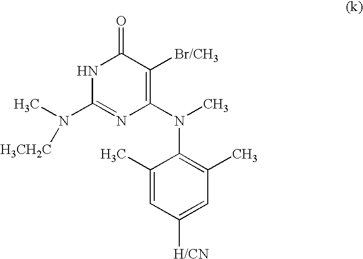
or a pharmaceutically acceptable salt, prodrug, stereoisomer, tautomer, N-oxide or quaternary amine thereof.

22. A compound of Formula (l): 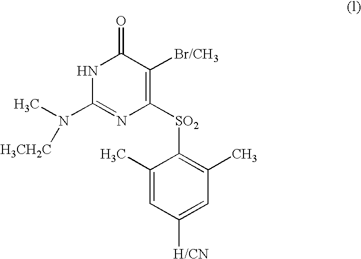
or a pharmaceutically acceptable salt, prodrug, stereoisomer, tautomer, N-oxide or quaternary amine thereof.

23. A compound of Formula (m): 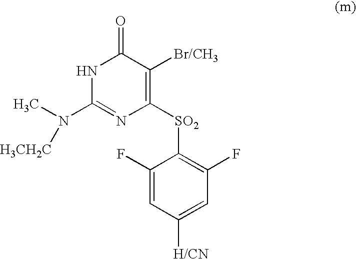
or a pharmaceutically acceptable salt, prodrug, stereoisomer, tautomer, N-oxide or quaternary amine thereof.

24. A compound of Formula (n): 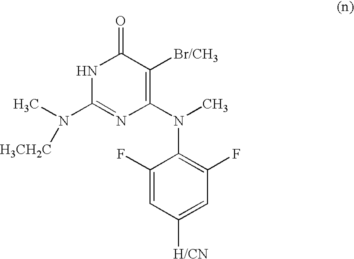
or a pharmaceutically acceptable salt, prodrug, stereoisomer, tautomer, N-oxide or quaternary amine thereof.

25. A compound of Formula (o): 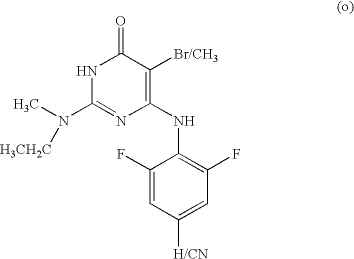
or a pharmaceutically acceptable salt, prodrug, stereoisomer, tautomer, N-oxide or quaternary amine thereof.

26. A compound of Formula (p): 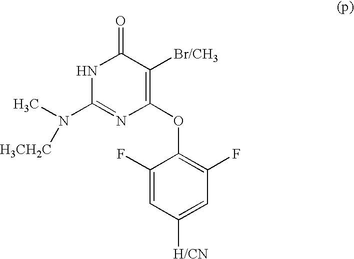
or a pharmaceutically acceptable salt, prodrug, stereoisomer, tautomer, N-oxide or quaternary amine thereof.

27. A compound of Formula (i): 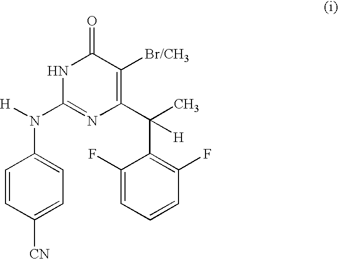
or a pharmaceutically acceptable salt, prodrug, stereoisomer, tautomer, N-oxide or quaternary amine thereof.

28. A compound of Formula (ii): 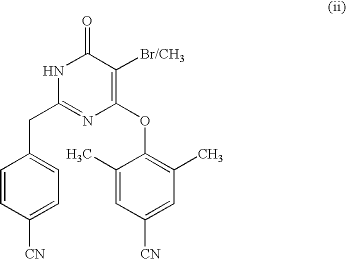
or a pharmaceutically acceptable salt, prodrug, stereoisomer, tautomer, N-oxide or quaternary amine thereof.

29. A compound of Formula (iii): 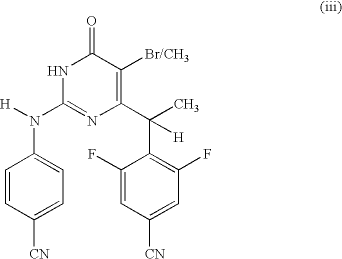
or a pharmaceutically acceptable salt, prodrug, stereoisomer, tautomer, N-oxide or quaternary amine thereof.

30. A compound of Formula (iv): 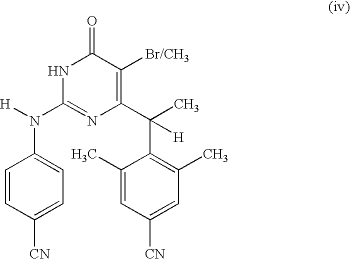
or a pharmaceutically acceptable salt, prodrug, stereoisomer, tautomer, N-oxide or quaternary amine thereof.

31. A compound of Formula (v): 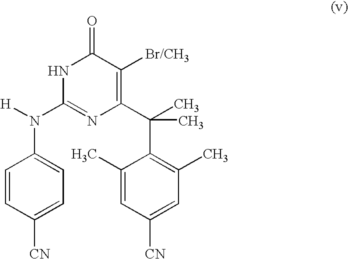
or a pharmaceutically acceptable salt, prodrug, stereoisomer, tautomer, N-oxide or quaternary amine thereof.

32. A compound of Formula (vi): 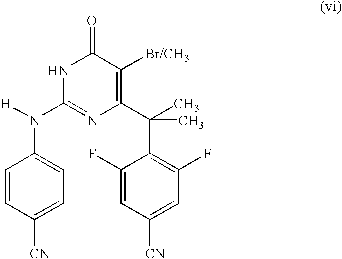
or a pharmaceutically acceptable salt, prodrug, stereoisomer, tautomer, N-oxide or quaternary amine thereof.

33. A compound of Formula (vii): 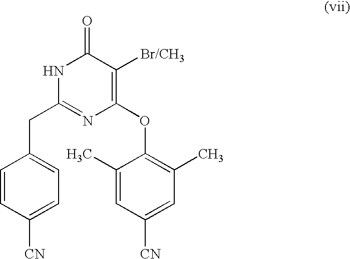
or a pharmaceutically acceptable salt, prodrug, stereoisomer, tautomer, N-oxide or quaternary amine thereof.

34. A compound of Formula (viii): 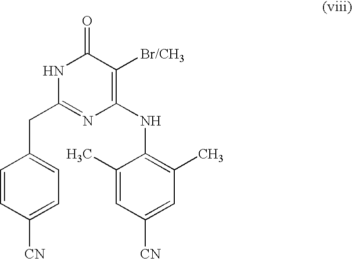
or a pharmaceutically acceptable salt, prodrug, stereoisomer, tautomer, N-oxide or quaternary amine thereof.

35. A compound of Formula (ix): 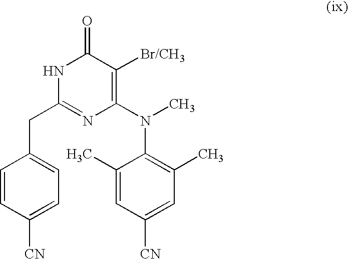
or a pharmaceutically acceptable salt, prodrug, stereoisomer, tautomer, N-oxide or quaternary amine thereof.

36. A compound of Formula (x): 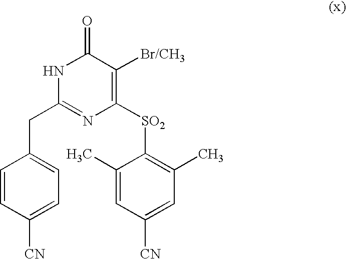
or a pharmaceutically acceptable salt, prodrug, stereoisomer, tautomer, N-oxide or quaternary amine thereof.

37. A pharmaceutical composition comprising an effective anti-HIV treatment amount of a compound of claim 1, or its pharmaceutically acceptable salt, prodrug, stereoisomer, tautomer, N-oxide or quaternary amine, optionally with a pharmaceutically acceptable carrier or diluent.

38. A pharmaceutical composition comprising an effective anti-HIV treatment amount of a compound of claim 1, or its pharmaceutically acceptable salt, prodrug, stereoisomer, tautomer, N-oxide or quaternary amine, in combination with at least one other anti-HIV agent, optionally with a pharmaceutically acceptable carrier or diluent.

39. The pharmaceutical composition of claim 38 wherein the other anti-HIV agent is a reverse transcriptase inhibitor.

40. The pharmaceutical composition of claim 39 wherein the reverse transcriptase inhibitor induces a mutation lysine 103→ asparagines and/or tyrosine 181→ cysteine in HIV reverse transcriptase.

41. A method for the treatment or prophylaxis of an HIV-infection in a host comprising administering to said host an anti-HIV effective treatment amount of a compound of claim 1, or its pharmaceutically acceptable salt, prodrug, stereoisomer, tautomer, N-oxide or quaternary amine, optionally with a pharmaceutically acceptable carrier or diluent.

42. The method of claim 41 wherein the host is a human.

43. A method for the treatment of prophylaxis of an HIV-infection in a host comprising administering to said host an anti-HIV effective treatment amount of a compound of claim 1, or its pharmaceutically acceptable salt, prodrug, stereoisomer, tautomer, N-oxide or quaternary amine, in combination and/or alternation with at least one other anti-HIV agent, optionally with a pharmaceutically acceptable carrier or diluent.

44. The method of claim 43 wherein the host is a human.

45. The method of claim 43 wherein the other anti-HIV agent is a reverse transcriptase inhibitor.

46. The method of claim 45 wherein the reverse transcriptase inhibitor induces a mutation lysine 103→ asparagines and/or tyrosine 181→ cysteine in HIV reverse transcriptase.

47. A method for the treatment or prophylaxis of an HIV-infection in a host, wherein the HIV has a mutation at lysine 103→ asparagines and/or tyrosine 181→ cysteine in HIV reverse transcriptase, comprising administering to said host an effective anti-HIV treatment amount of a compound according to claim 1, or its pharmaceutically acceptable salt, prodrug, stereoisomer, tautomer, N-oxide or quaternary amine, optionally with a pharmaceutically acceptable carrier or diluent.

48. The method of claim 47 wherein the host is a human.

49. A method for the treatment or prophylaxis of an HIV-infection in a host, wherein the HIV has a mutation at lysine 103→ asparagines and/or tyrosine 181→ cysteine in HIV reverse transcriptase, comprising administering to said host an effective anti-HIV treatment amount cf a compound according to claim 1, or its pharmaceutically acceptable salt, prodrug, stereoisomer, tautomer, N-oxide or quaternary amine, in combination and/or alternation with at least one other anti-HIV agent, optionally with a pharmaceutically acceptable carrier or diluent.

50. The method of claim 49 wherein the host is a human.

51. A method for the treatment or prophylaxis of an HIV-infection in a host wherein the HIV is resistant to one or more reverse transcriptase inhibitor(s), comprising administering to said host an anti-HIV effective treatment amount of a compound of claim 1, or its pharmaceutically acceptable salt, prodrug, stereoisomer, tautomer, N-oxide or quaternary amine, optionally with a pharmaceutically acceptable carrier or diluent.

52. The method of claim 51 wherein the host is a human.

53. A method for the treatment or prophylaxis of an HIV-infection in a host wherein the HIV is resistant to one or more reverse transcriptase inhibitor(s), comprising administering to said host an anti-HIV effective treatment amount of a compound of claim 1, or its pharmaceutically acceptable salt, prodrug, stereoisomer, tautomer, N-oxide or quaternary amine, in combination and/or alternation with at least one other anti-HIV agent, optionally with a pharmaceutically acceptable carrier or diluent.

54. The method of claim 53 wherein the host is a human.

55. A method for salvage therapy in the treatment or prophylaxis of an anti-HIV infection in a host comprising administering to said host an anti-HIV effective treatment amount of a compound of claim 1, or its pharmaceutically acceptable salt, prodrug, stereoisomer, tautomer, N-oxide or quaternary amine, optionally with a pharmaceutically acceptable carrier or diluent.

56. The method of claim 55 wherein the host is a human.

57. A method for salvage therapy in the treatment or prophylaxis of an anti-HIV infection in a host comprising administering to said host an anti-HIV effective treatment amount of a compound of claim 1, or its pharmaceutically acceptable salt, prodrug, stereoisomer, tautomer, N-oxide or quaternary amine, in combination and/or alternation with at least one other anti-HIV agent, optionally with a pharmaceutically acceptable carrier or diluent.

58. The method of claim 57 wherein the host is a human.

59. A pharmaceutical composition comprising an effective anti-HIV treatment amount of a compound of claim 6, or its pharmaceutically acceptable salt, prodrug, stereoisomer, tautomer, N-oxide or quaternary amine, optionally with a pharmaceutically acceptable carrier or diluent.

60. A pharmaceutical composition comprising an effective anti-HIV treatment amound of a compound of claim 6, or its pharmaceutically acceptable salt, prodrug, stereoisomer, tautomer, N-oxide or quaternary amine, in combination and/or alternation with at least one other anti-HIV agent, optionally with a pharmaceutically acceptable carrier or diluent.

61. The pharmaceutical composition of claim 60 wherein the other anti-HIV agent is a reverse transcriptase inhibitor.

62. The pharmaceutical composition of claim 61 wherein the reverse transcriptase inhibitor induces a mutation lysine 103→ asparagines and/or tyrosine 181→ cysteine in HIV reverse transcriptase.

63. A method for the treatment or prophylaxis of an HIV-infection in a host comprising administering to said host an anti-HIV effective treatment amount of a compound of claim 6, or its pharmaceutically acceptable salt, prodrug, stereoisomer, tautomer, N-oxide or quaternary amine, optionally with a pharmaceutically acceptable carrier or diluent.

64. The method of claim 63 wherein the host is a human.

65. A method for the treatment or prophylaxis of an HIV-infection in a host comprising administering to said host an anti-HIV effective treatment amount of a compound of claim 6, or its pharmaceutically acceptable salt, prodrug, stereoisomer, tautomer, N-oxide or quaternary amine, in combination and/or alternation with at least one other anti-HIV agent, optionally with a pharmaceutically acceptable carrier or diluent.

66. The method of claim 65 wherein the host is a human.

67. The method of claim 65 wherein the other anti-HIV agent is a reverse transcriptase inhibitor.

68. The method of claim 67 wherein the reverse transcriptase inhibitor induces a mutation lysine 103→ asparagines and/or tyrosine 181→ cysteine in HIV reverse transcriptase.

69. A method for the treatment or prophylaxis of an HIV-infection in a host, wherein the HIV has a mutation at lysine 103→ asparagines and/or tyrosine 181→ cysteine in HIV reverse transcriptase, comprising administering to said host an effective anti-HIV treatment amount of a compound according to claim 6, or its pharmaceutically acceptable salt, prodrug, stereoisomer, tautomer, N-oxide or quaternary amine, optionally with a pharmaceutically acceptable carrier or diluent.

70. The method of claim 69 wherein the host is a human.

71. A method for the treatment or prophylaxis of an HIV-infection in a host, wherein the HIV has a mutation at lysine 103→ asparagines and/or tyrosine 181→ cysteine in HIV reverse transcriptase, comprising administering to said host an effective anti-HIV treatment amount of a compound according to claim 6, or its pharmaceutically acceptable salt, prodrug, stereoisomer, tautomer, N-oxide or quaternary amine, in combination and/or alternation with at least one other anti-HIV agent, optionally with a pharmaceutically acceptable carrier or diluent.

72. The method of claim 71 wherein the host is a human.

73. A method for the treatment or prophylaxis of an HIV-infection in a host wherein the HIV is resistant to one or more reverse transcriptase inhibitor(s), comprising administering to said host an anti-HIV effective treatment amount of a compound of claim 6, or its pharmaceutically acceptable salt, prodrug, stereoisomer, tautomer, N-oxide or quaternary amine, optionally with a pharmaceutically acceptable carrier or diluent.

74. The method of claim 73 wherein the host is a human.

75. A method for the treatment or prophylaxis of an FRV-infection in a host wherein the HIV is resistant to one or more reverse transcriptase inhibitor(s), comprising administering to said host an anti-HIV effective treatment amount of a compound of claim 6, or its pharmaceutically acceptable salt, prodrug, stereoisomer, tautomer, N-oxide or quaternary amine, in combination and/or alternation with at least one other anti-HIV agent, optionally with a pharmaceutically acceptable carrier or diluent.

76. The method of claim 75 wherein the host is a human.

77. A method for salvage therapy in the treatment or prophylaxis of an anti-HIV infection in a host comprising administering to said host an anti-HIV effective treatment amount of a compound of claim 6, or its pharmaceutically acceptable salt, prodrug, stereoisomer, tautomer, N-oxide or quaternary amine, optionally with a pharmaceutically acceptable carrier or diluent.

78. The method of claim 77 wherein the host is a human.

79. A method for salvage therapy in the treatment or prophylaxis of an anti-HIV infection in a host comprising administering to said host an anti-HIV effective treatment amount of a compound of claim 6, or its pharmaceutically acceptable salt, prodrug, stereoisomer, tautomer, N-oxide or quaternary amine, in combination and/or alternation with at least one other anti-HIV agent, optionally with a pharmaceutically acceptable carrier or diluent.

80. The method of claim 79 wherein the host is a human.
